(19) United States
(12) Patent Application Publication
(10) Pub. No.: US 2005/0187278 A1
(43) Pub. Date: Aug. 25, 2005

(54) Treatment or prevention of vascular disorders with Cox-2 inhibitors in combination with cyclic AMP-specific phosphodiesterase inhibitors

(75) Inventor: Duncan P. Taylor, Bridgewater, NJ (US)

Correspondence Address: James E. Davis;Harness, Dickey & Pierce, P.L.C., 7700 Bonhomme, Suite 400, Clayton, MO, 63105, US

(73) Assignee: Pharmacia Corporation, Chesterfield, MO (US)

(21) Appl. No.: 10/927,198

(22) Filed: Aug. 26, 2004

Related U.S. Application Data
(60) Provisional application No. 60/498,529, filed on Aug. 28, 2003.
(60) Provisional application No. 60/513,099, filed on Oct. 21, 2003.

(51) Int. Cl. A61K 31/415; A61K 31/18

(52) U.S. Cl. 514/406; 514/471; 514/602

(57) ABSTRACT

A method is described for the prevention and/or treatment of vascular disorders and vascular disorder-related complications in a subject, the method comprising administering to the subject a Cox-2 inhibitor in combination with a cAMP-specific PDE inhibitor. Also described are therapeutic and pharmaceutical compositions and kits that are useful in the present invention.

### CROSS-REFERENCE TO RELATED APPLICATIONS

This application is related to and claims the priority benefit of U.S. Provisional Patent Application Ser. No. 60/498,529 filed Aug. 28, 2003 and U.S. Provisional Patent Application Ser. No. 60/513,099 filed Oct. 21, 2003, both of which are incorporated by reference herein in their entirety.

### BACKGROUND OF THE INVENTION

(1) Field of the Invention

The present invention relates to the prevention or treatment of vascular disorders, and more particularly to the prevention and treatment of vascular disorders and vascular disorder-related complications, by pharmacologic interventions.

(2) Description of the Related Art

Vascular disorders such as cardiovascular disease are responsible for every 1 in 2.5 deaths in the United States, claiming more lives each year than the next five leading causes of death combined, those being cancer, chronic lower respiratory disease, accidents, diabetes, and influenza/pneumonia. In the United States, there is an average of one death every 33 seconds that is attributed to cardiovascular disease. See American Heart Association. Heart Disease and Stroke Statistics—2003 Update. Dallas, Tex.: American Heart Association; 2002.

Cardiovascular diseases include coronary heart disease and heart attack, angina pectoris, stroke, transient ischemic attack, high blood pressure, arrhythmias, including atrial fibrillation and flutter, tachycardia, and ventricular fibrillation, diseases of the arteries, including atherosclerosis, peripheral arterial disease, aortic aneurysm, and deep vein thrombosis, bacterial endocarditis, cardiomyopathy, congenital cardiovascular defects, congestive heart failure, rheumatic heart disease, and valvular heart disease. A subset of these diseases falls into the category of disorders that are treated with hematological drugs, such as thrombolytic drugs, anti-platelet drugs, and anticoagulants. Disorders of this classification include coronary heart disease, heart attack, stroke, transient ischemic attack, high blood pressure, atherosclerosis, peripheral arterial disease, aortic aneurysm, and deep vein thrombosis. For disorders such as coronary heart disease, high blood pressure, and atherosclerosis, the hematological drug is often administered in conjunction with a vasodilator to further improve blood flow and decrease blood pressure.

Traditionally, the most commonly prescribed anti-platelet drug for the indications described above has been aspirin, a non-steroidal anti-inflammatory drug (NSAID). It is now recognized that many of the traditional NSAIDs are inhibitors of two cyclooxygenases, cyclooxygenase-1 (Cox-1) and cyclooxygenase-2 (Cox-2). These two enzymes are involved in the critical initiation step of prostaglandin synthesis; the addition of molecular oxygen to arachidonic acid in the cell membrane. See Needleman, P. et al., Annu Rev Biochem, 55:69-102 (1986).

Cox-1 is constitutively active and is responsible for the synthesis of housekeeping prostaglandins critical to maintaining normal renal function, gastric mucosal integrity, and vascular homeostasis. Cox-2 expression is induced by cytokines and growth factors in inflammatory cells, leading to the release of prostanoids (prostaglandin E2) which sensitize peripheral nociceptor terminals and produce localized pain hypersensitivity, inflammation, and edema. See e.g. Samad, T. A. et al., Nature 410:471-5 (2001). Because many common NSAIDs inhibit prostaglandin synthesis by blocking the activity of both Cox-1 and Cox-2, side effects associated with long-term administration of these drugs such as gastrointestinal bleeding and ulcers are thought to be a result of inhibiting the homeostatic functions of Cox-1, while the inhibiton of Cox-2 accounts for their analgesic properties.

Research into the area of arachidonic acid metabolism has resulted in the discovery of compounds that inhibit the Cox-2 enzyme to a greater extent than the activity of Cox-1. The Cox-2 selective inhibitors are believed to offer advantages that include the capacity to prevent or reduce inflammation while avoiding harmful side effects associated with the inhibition of Cox-1. Thus, Cox-2 selective inhibitors have shown great promise for use in therapies, especially in therapies that require maintenance administration.

The anti-platelet effects of aspirin are mediated through the inhibition of the cyclooxygenase enzymes, which catalyze the synthesis of eicosanoids that are critical for platelet-vessel wall interactions. Specifically, aspirin exerts its anti-platelet effects through the inhibition of Cox-1-induced production of thromboxane A₂, involved in platelet aggregation. See Catella-Lawson, F., Neurology, 57:S5-7 (2001). Both the presence and the biological significance of the Cox-2 enzyme in platelets has been somewhat controversial. Recent studies indicate that Cox-2 expression is limited to newly formed platelets, apparently transferred from parental megakaryocyte cells, and is not involved in platelet aggregation. See Rocca, B. et al., PNAS, 99:7634-39 (2002). Therefore, while the effects of Cox-2 inhibitors on inflammation and inflammation-related disorders have been relatively widely recognized, the significance of these selective inhibitors in treating vascular disorders related to inappropriate platelet aggregation remain unknown.

One specific example of a disorder related to platelet aggregation is peripheral arterial disease. The primary symptom of peripheral arterial disease is intermittent claudication, which is defined as leg muscle pain, cramping, and fatigue associated with exercise. The recently approved drug cilostazol (Pletal®) has been effectively used in the treatment of this disorder. Cilostazol is a phosphodiesterase 3 inhibitor, as well as an activator of the enzyme lipoprotein lipase. See Doggrell, S. A., Expert Opin Pharmacother, 2:1725-36 (2001).

The phosphodiesterases (“PDEs”) comprise a large family of enzymes that catalyze the hydrolysis of the intracellular second messenger cyclic nucleotides, cAMP and cGMP, to their biologically inactive forms, 5′AMP and 5′GMP. In conjunction with adenylyl and guanylyl cyclases, PDEs are able to regulate cell signaling mechanisms that are mediated by cAMP and cGMP by reducing available intracellular pools. These second messengers play a critical role in the transduction of extracellular signals to intracellular compartments.

Eleven distinct classes of PDEs have been identified, each with unique catalytic properties, substrate specificities, and tissue expression patterns. See Uckert, S., et al., World J Urol, 19:14-22 (2001). In the course of the discovery of these different families, selective inhibitors with selectivity for specific PDE classes have been designed and synthesized. PDE 3 and PDE 4 have been identified as having specific catalytic activity for cAMP, therefore the cAMP-specific PDE inhibitors targeting these particular isoenzymes result in the accumulation of active cAMP in the cell. This leads to an induction of cAMP-dependent protein kinase (protein kinase A or PKA) activity and transcription of cAMP-inducible genes, which play an important role in controlling the proliferation and differentiation of several cell types. In addition to its ability to hydrolyze cAMP, PDE 3 is further characterized as a cGMP-inhibited cAMP-specific phosphodiesterase, since cGMP acts as a competitive substrate for PDE 3, blocking its hydrolysis of cAMP. PDE 3 is also the major cardiovascular phosphodiesterase.

The therapeutic effects of the cAMP-specific PDE 3 inhibitor cilostazol in the treatment of peripheral arterial disease appear to be mediated by increasing intracellular levels of cAMP and consequently inhibiting the activation of platelets, as well as by promoting vasodilation. See Tanaka, K., et al., Stroke, 20:668-73 (1989). However, it has not been reported whether other cAMP-specific PDE inhibitors might have the same beneficial effect in the treatment of other vascular disorders. Additionally, as mentioned previously, it has been shown that NSAIDs such as aspirin have been used in the past for treating certain vascular disorders, but it has not been reported whether a combination of a cAMP-specific PDE inhibitor and anti-inflammatory compounds such as Cox-2 inhibitors would be useful in the treatment of vascular disorders such as stroke, ischemic attack, deep vein thrombosis, and coronary heart disease.

While the effects of Cox-2 inhibitors on inflammation and inflammation-related disorders have been relatively widely recognized, the effects of Cox-2 inhibitors on cerebrovascular and cardiovascular diseases and disorders have not been as widely reported. U.S. Pat. No. 6,323,226 to Delgado III, et al. teaches the use of a Cox-2 selective inhibitor in the treatment of congestive heart failure. U.S. Pat. No. 6,245,797 to Winokur discloses the combination of Cox-2 inhibitors and an HMG-COA reductase inhibitor for the treatment of atherosclerosis. U.S. Pat. Nos. 6,136,804 and 6,511,968, both to Nichtberger, relate to the use of Cox-2 inhibitors in combination with anti-platelet drugs in the treatment of acute coronary ischemia.

Although the conventional treatment approaches to vascular disorders described above have been beneficial, the extremely high incidence of mortality associated with cardiovascular disorders indicates that improved treatments are needed. Therefore, the present invention addresses this need by providing a combination therapy comprised of a cAMP-specific PDE inhibitor and a Cox-2 selective inhibitor for the treatment of vascular disorders, and specifically cerebrovascular and cardiovascular disorders.

### SUMMARY

Briefly, therefore, the present invention is directed to a novel method for the prevention or treatment of vascular disorders in a subject comprising administering to the subject a cAMP-specific PDE inhibitor in combination, with a Cox-2 inhibitor.

The present invention is also directed to a novel method for the prevention or treatment of vascular disorders in a subject in need of such prevention or treatment, the method comprising administering to the subject a cAMP-specific PDE inhibitor in combination with a Cox-2 inhibitor.

The present invention is also directed to a novel composition comprising at least one Cox-2 inhibitor and at least one cAMP-specific PDE inhibitor.

The present invention is also directed to a novel pharmaceutical composition comprising at least one Cox-2 inhibitor, at least one cAMP-specific PDE inhibitor, and a pharmaceutically-acceptable excipient.

The present invention is also directed to a novel kit comprising a first dosage form comprising a Cox-2 inhibitor and a second dosage comprising a cAMP-specific PDE inhibitor.

Among the several advantages found to be achieved by the present invention, therefore, may be noted the provision of a method and compositions for the treatment of vascular disorders, and in particular, for cerebrovascular and cardiovascular disorders, and the provision of such method and compositions that are efficacious, safe, and easy to administer.

### DETAILED DESCRIPTION OF THE PREFERRED EMBODIMENTS

In accordance with the present invention, it has been discovered that vascular disorders, and in particular, cerebrovascular and cardiovascular disorders may be treated and/or prevented in a subject by administering to the subject a Cox-2 inhibitor in combination with a cAMP-specific PDE inhibitor. In preferred embodiments, the method is useful for the purpose of preventing and treating vascular disorders in a subject that is in need of such prevention or treatment.

The methods and compositions of the present invention would be useful, for example, to reduce such vascular disorder symptoms as hypertension and ischemia in a subject suffering from such symptoms. The combination therapy of the present invention would also be useful to prevent the occurrence of such symptoms.

The combination therapy of the present invention is also useful for the treatment of vascular disorder-related complications, which may arise indirectly from having a vascular disorder, by treating the underlying vascular disorder itself or a symptom thereof. For example, if a subject is suffering from a vascular disorder-related complication, such as a heart failure, the treatment of the underlying vascular disorder symptom, such as hypertension, by the methods and compositions of the present invention will likewise improve the symptoms of the associated complication.

The methods and compositions of the present invention are also useful to reduce the number of hospitalizations of subjects suffering from vascular disorders, or to prevent or retard, in subjects, the development of complications associated with vascular disorders, such as, for example, heart failure, which may eventually arise from having a vascular disorder.

The administration of a Cox-2 inhibitor in combination with a cAMP-specific PDE inhibitor for the prevention or treatment of vascular disorders and vascular disorder-related complications is an unexpectedly effective treatment and preventative therapy. Such administration is effective for improving the symptoms of vascular disorders and vascular disorder-related complications while avoiding or reducing certain disadvantages of conventional treatment agents. As used herein, the terms “conventional treatment agent” or “conventional treatment agents” refers to any compound that is other than a Cox-2 inhibitor or a cAMP-specific PDE inhibitor.

Combination therapies comprising Cox-2 inhibitors and cAMP-specific PDE inhibitors are useful not only for improving vascular disorder symptoms and shortening recovery times, but also for reducing the dosages of conventional treatment agents that are normally required. Reduced dosages of conventional treatment agents are beneficial where normal dosages exhibit harmful side effects or require burdensome treatment regimens. The administration of low dosages of conventional treatment agents can, in one embodiment, provide a reduction in side effects corresponding to such agents.

As used herein, the terms “lowered dosages”, “low dose”, or “low dose amount”, in characterizing a therapeutically effective amount of a Cox-2 inhibitor in combination with a cAMP-specific PDE inhibitor defines a quantity of such agent, or a range of quantity of such agent, that is capable of preventing or treating the symptoms of a vascular disorder or a vascular disorder-related complication while optionally reducing or avoiding one or more side effects of a monotherapy with a conventional cAMP-specific PDE inhibitor.

The combination therapy of a Cox-2 inhibitor and a cAMP-specific PDE inhibitor may also be useful for decreasing the required number of separate dosages, thus, potentially improving patient compliance.

The administration of a Cox-2 inhibitor in combination with a cAMP-specific PDE inhibitor is an effective treatment for vascular disorders and vascular disorder-related complications, and in preferred embodiments, is superior to the use of either agent alone. Moreover, in preferred embodiments, the combination therapies of the present invention demonstrate a synergistic efficacy for treating and preventing vascular disorders and vascular disorder-related complications that is greater than what would be expected from simply combining any of the individual monotherapies. As used herein, the term “synergistic” refers to the combination of a Cox-2 inhibitor and a cAMP-specific PDE inhibitor as a combined therapy having an efficacy for the prevention and treatment of vascular disorders that is greater than what would be expected merely from the sum of their individual effects. The synergistic effects of the embodiments of the present invention's combination therapies encompass additional unexpected advantages for the treatment and prevention of vascular disorders. Such additional advantages include, but are not limited to, lowering the required dose of cAMP-specific PDE inhibitors, reducing the side effects of conventional treatment agents, and rendering those agents more tolerable to subjects in need of vascular disorder therapy.

As used herein, the term “monotherapies” is intended to embrace administration of a Cox-2 inhibitor to a subject suffering from a vascular disorder or vascular disorder-related complication as a single therapeutic treatment without an additional therapeutic treatment comprising a cAMP-specific PDE inhibitor and vice versa. As used herein, the terms “combination therapy”, “co-administration”, “co-administering”, “administration with”, “administering”, “combination”, or “co-therapy”, when referring to the use of a Cox-2 inhibitor in combination with a cAMP-specific PDE inhibitor, are intended to embrace administration of each agent in a sequential manner in a regimen that will provide beneficial effects of the drug combination, and is intended as well to embrace co-administration of these agents in a substantially simultaneous manner.

Substantially simultaneous administration can be accomplished, for example, by administering to the subject the Cox-2 inhibitor in combination with a cAMP-specific PDE inhibitor, together in one therapeutic dosage form, such as in a single capsule, tablet, or injection, or in multiple separate therapeutic dosage forms, such as in separate capsules, tablets, or injections.

Sequential or substantially simultaneous administration of each therapeutic agent can be effected by any appropriate route including, but not limited to, oral routes, intravenous routes, intramuscular routes, subcutaneous routes, intraarticular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

The phrase “combination therapy” also can embrace the administration of the therapeutic agents as described above in further combination with other biologically active ingredients and non-drug therapies.

Sequential administration of such treatments encompasses both relatively short and relatively long periods between the administration of each of the compounds of the present method. However, for purposes of the present invention, the cAMP-specific PDE inhibitor is administered while the Cox-2 inhibitor is still having an efficacious effect on the subject.

Preferably, the cAMP-specific PDE inhibitor is to be given to the subject within the therapeutic response time of the administered Cox-2 inhibitor. As used herein, the terms “therapeutic response time” mean the duration of time after administration that a compound has a therapeutic effect within a subject's body.

As used herein, the terms “therapeutically effective” are intended to qualify the amount of an agent for use in a therapy that will achieve the goal of preventing or treating by improvement in the severity of the vascular disorder symptoms or vascular disorder-related complication symptoms in a subject, while avoiding adverse side effects typically associated with conventional treatment agents.

In one embodiment, the present invention encompasses a method for preventing vascular disorders or vascular disorder-related complications in a subject, and in preferred embodiments, preventing vascular disorders or vascular disorder-related complications in subject that is predisposed to vascular disorders or vascular disorder-related complications, the method comprising administering to the subject a Cox-2 inhibitor in combination with a cAMP-specific PDE inhibitor.

As used herein, the terms “to prevent”, “preventing”, or “prevention” refer to any reduction, no matter how slight, of a subject's predisposition or risk for developing a vascular disorder or a vascular disorder-related complication. For purposes of prevention, the subject is any subject, and preferably is a subject that is at risk for, or is predisposed to, developing a vascular disorder or a vascular disorder-related complication. As used herein, the terms “predisposition”, “predisposed to” or “at risk for,” all of which may be used interchangeably herein, includes any subject with an increased chance for developing a vascular disorder or a vascular disorder-related complication. The subject may be at risk due to genetic predisposition, trauma, sex, age, exposure to vascular disorder causing agents, and the like. The subject may also be at risk for re-developing a vascular disorder or a vascular disorder-related complication after suffering from a vascular disorder or a vascular disorder-related complication. The subject may also be at risk due to lifestyle, diet and/or physiological factors such as anatomical and biochemical abnormalities.

In another embodiment, the present invention encompasses a method for treating vascular disorders or vascular disorder-related complications in a subject, and in preferred embodiments, treating vascular disorders or vascular disorder-related complications in subject that is suffering from a vascular disorder or a vascular disorder-related complication, the method comprising administering to the subject a Cox-2 inhibitor in combination with a cAMP-specific PDE inhibitor.

As used herein, the terms “treating”, “treatment”, “treated”, or “to treat,” mean to alleviate symptoms, eliminate the causation either on a temporary or permanent basis, or to alter or slow the appearance of symptoms or symptom worsening.

The amount of the Cox-2 inhibitor that is used in the method is selected so that it constitutes a vascular disorder treatment or prevention effective amount. In some embodiments, the amount of cAMP-specific PDE inhibitor is selected so that when they are used in combination with the Cox-2 inhibitor, a dosage of the combination provides a vascular disorder treatment or prevention effective amount.

A component of the present invention is a Cox-2 inhibitor.

Inhibitors of the Cox pathway in the metabolism of arachidonic acid may inhibit enzyme activity through a variety of mechanisms. By way of example, the Cox-2 inhibitors used in the methods described herein may block the enzyme activity directly by binding at the substrate site of the enzyme. In preferred embodiments, the use of a Cox-2 selective inhibitor is highly advantageous in that it minimizes the gastric side effects that can occur with non-selective non-steroidal anti-inflammatory drugs (NSAIDs), especially where prolonged treatment is expected.

The terms “cyclooxygenase-2 inhibitor”, or “Cox-2 inhibitor”, which can be used interchangeably herein, embrace compounds, which inhibit the Cox-2 enzyme regardless of the degree of inhibition of the Cox-1 enzyme, and include pharmaceutically acceptable salts of those compounds. Thus, for purposes of the present invention, a compound is considered a Cox-2 inhibitor irrespective of whether the compound inhibits the Cox-2 enzyme to an equal, greater, or lesser degree than the Cox-1 enzyme.

In one embodiment of the present invention, it is preferred that the Cox-2 inhibitor compound is a non-steroidal anti-inflammatory drug (NSAID). Therefore, preferred materials that can serve as the Cox-2 inhibitor of the present invention include non-steroidal anti-inflammatory drug compounds, a pharmaceutically acceptable salt thereof, mixed isomer, or a pure (−) or (+) optical isomeric form thereof.

Examples of NSAID compounds that are useful in the present invention include acemetacin, acetyl salicylic acid, alclofenac, alminoprofen, azapropazone, benorylate, benoxaprofen, bucloxic acid, carprofen, choline magnesium trisalicylate, clidanac, clopinac, dapsone, diclofenac, diflunisal, droxicam, etodolac, fenoprofen, fenbufen, fenclofenec, fentiazac, floctafenine, flufenisal, flurbiprofen, (r)-flurbiprofen, (s)-flurbiprofen, furofenac, feprazone, flufenamic acid, fluprofen, ibufenac, ibuprofen, indometacin, indomethacin, indoprofen, isoxepac, isoxicam, ketoprofen, ketorolac, miroprofen, piroxicam, meloxicam, mefenamic, mefenamic acid, meclofenamic acid, meclofen, nabumetone, naproxen, niflumic acid, nitroflurbiprofen, oxaprozin, oxipinac, oxyphenbutazone, phenylbutazone, podophyllotoxin derivatives, proglumetacin, piprofen, pirprofen, prapoprofen, salicylic acid, salicylate, sudoxicam, suprofen, sulindac, tenoxicam, tiaprofenic acid, tiopinac, tioxaprofen, tolfenamic acid, tolmetin, zidometacin, zomepirac, and 2-fluoro-a-methyl[1,1′-biphenyl]-4-acetic acid, a 4-(nitrooxy)butyl ester, and mixtures thereof.

Further preferred NSAID compounds include ibuprofen, naproxen, sulindac, ketoporfen, fenoprofen, tiaprofenic acid, suprofen, etodolac, carprofen, ketrolac, piprofen, indoprofen, salicylic acid, flurbiprofen, and mixtures thereof.

In a preferred embodiment, the Cox-2 inhibitor is a Cox-2 selective inhibitor. The term “Cox-2 selective inhibitor” embraces compounds, which selectively inhibit the Cox-2 enzyme over the Cox-1 enzyme, and also include pharmaceutically acceptable salts and prodrugs of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1|C₅₀/Cox-2|C₅₀). A Cox-2 selective inhibitor is any inhibitor for which the ratio of Cox-1|C₅₀ to Cox-2|C₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of Cox activity. Preferred Cox-2 selective inhibitors of the present invention have a Cox-2 IC₅₀ of less than about 1 μM, more preferred of less than about 0.5 μM, and even more preferred of less than about 0.2 μM.

Preferred Cox-2 selective inhibitors have a Cox-1|C₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of Cox-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic Cox-2 selective inhibitor valdecoxib. An example of a preferred Cox-2 selective inhibitor prodrug is sodium parecoxib. A class of prodrugs of Cox-2 inhibitors is described in U.S. Pat. No. 5,932,598.

In one embodiment of the present invention, the Cox-2 selective inhibitor is of the chromene/chroman (“chromene”) structural class, which encompasses substituted benzopyrans or substituted benzopyran analogs, as well as substituted benzothiopyrans, dihydroquinolines, or dihydronaphthalenes having the structure of general Formula I, shown below, and including, by way of non-limiting example, the chromene compounds described below, and the diastereomers, enantiomers, racemates, tautomers, salts, esters, amides and prodrugs thereof.

Chromenes that can serve as a Cox-2 selective inhibitor of the present invention include any one or more of the compounds that are described in U.S. Pat. Nos. 6,271,253; 6,492,390; 6,034,256 and 6,077,850. One such class of compounds is defined by the general formula shown below in formula I: 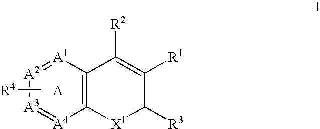
wherein X¹ is selected from O, S, CR^{c}R^{b} and NR^{a};
wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b}R^{c} forms a cycloalkyl ring;
wherein R¹ is selected from carboxyl, alkyl, aralkyl, aminocarbonyl, C₁-C₆-alkylsulfonylaminocarbonyl and alkoxycarbonyl;
wherein R² is selected from hydrido, phenyl, thienyl, C₂-C₆-alkynyl, C₁-C₆-alkyl and C₂-C₆-alkenyl;
wherein R³ is selected from C₁-C₃-perfluoroalkyl, chloro, C₁-C₆-alkylthio, C₁-C₆-alkoxy, nitro, phenyl, cyano, cyano-C₁-C₃-alkyl, haloalkyl, alkyl, aralkyl, cycloalkyl, and aryl, wherein haloalkyl, alkyl, aralkyl, cycloalkyl, and aryl each is independently optionally substituted with one or more radicals selected from alkylthio, nitro and alkylsulfonyl;
wherein R⁴ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, O(CF₂)₂O—, aryloxy, arylthio, arylsulfinyl, heteroaryloxy, aralkyloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₁-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon; or
wherein R⁴ together with ring A forms a radical selected from naphthyl, quinolyl, isoquinolyl, quinolizinyl, quinoxalinyl and dibenzofuryl; or an isomer or pharmaceutically acceptable salt thereof.

The meaning of any substituent at any one occurrence in any general chemical formula herein, is independent of its meaning, or any other substituent's meaning, at any other occurrence, unless specified otherwise.

The term “alkyl” is used, either alone or within other terms such as “haloalkyl” and “alkylsulfonyl”; it embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. The number of carbon atoms can also be expressed as “C₁-C₅”, for example. Examples of such radicals include methyl, ethyl, n-propyl, isopropyl, n-butyl, isobutyl, sec-butyl, tert-butyl, pentyl, isoamyl, hexyl, octyl and the, like. The term “alkenyl” refers to an unsaturated, acyclic hydrocarbon radical, linear or branched, in so much as it contains at least one double bond. Unless otherwise noted, such radicals preferably contain from 2 to about 6 carbon atoms, preferably from 2 to about 4 carbon atoms, more preferably from 2 to about 3 carbon atoms. The alkenyl radicals may be optionally substituted with groups as defined below. Examples of suitable alkenyl radicals include propenyl, 2-chloropropylenyl, buten-1yl, isobutenyl, penten-1yl, 2-methylbuten-1-yl, 3-methylbuten-1-yl, hexen-1-yl, 3-hydroxyhexen-1-yl, hepten-1-yl, octen-1-yl, and the like. The term “alkynyl” refers to an unsaturated, acyclic hydrocarbon radical, linear or branched, in so much as it contains one or more triple bonds, such radicals preferably containing 2 to about 6 carbon atoms, more preferably from 2 to about 3 carbon atoms. The alkynyl radicals may be optionally substituted with groups as described below. Examples of suitable alkynyl radicals include ethynyl, proynyl, hydroxypropynyl, butyn-1-yl, butyn-2-yl, pentyn-1-yl, pentyn-2-yl, 4-methoxypentyn-2-yl, 3-methylbutyn-1-yl, hexyl-1-yl, hexyn-2-yl, hexyn-3-yl, 3,3-dimethylbutyn-1-yl radicals, and the like.

The term “oxo” means a single double-bonded oxygen. The terms “hydrido”, “—H”, or “hydrogen”, denote a single hydrogen atom (H). This hydrido radical may be attached, for example, to an oxygen atom to form a hydroxyl radical, or two hydrido radicals may be attached to a carbon atom to form a methylene (—CH₂—) radical.

The term “halo” means halogens such as fluorine, chlorine, and bromine or iodine atoms. The term “haloalkyl” embraces radicals wherein any one or more of the alkyl carbon atoms is substituted with halo as defined above. Specifically embraced are monohaloalkyl, dihaloalkyl, and polyhaloalkyl radicals. A monohaloalkyl radical, for one example, may have a bromo, chloro, or a fluoro atom within the radical. Dihalo radicals may have two or more of the same halo atoms or a combination of different halo radicals and polyhaloalkyl radicals may have more than two of the same halo atoms or a combination of different halo radicals. Likewise, the term “halo”, when it is appended to alkenyl, alkynyl, alkoxy, aryl, cycloalkyl, heteroalkyl, heteroaryl, and the like, includes radicals having mono-, di-, or tri-, halo substitution on one or more of the atoms of the radical.

The term “hydroxyalkyl” embraces linear or branched alkyl radicals having one to about ten carbon atoms any one of which may be substituted with one or more hydroxyl radicals.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and diaikoxyalkyl radicals. The “alkoxy” or “alkoxyalkyl” radicals may be further substituted with one or more halo atoms, such as fluoro, chloro, or bromo, to provide “haloalkoxy” or “haloalkoxyalkyl” radicals. Examples of “alkoxy” radicals include methoxy, butoxy, and trifluoromethoxy. Terms such as “alkoxy(halo)alkyl”, indicate a molecule having a terminal alkoxy that is bound to an alkyl, which is bonded to the parent molecule, while the alkyl also has a substituent halo group in a non-terminal location. In other words, both the alkoxy and the halo group are substituents of the alkyl chain.

The term “aryl”, alone or in combination, means a carbocyclic aromatic system containing one, two, or three rings wherein such rings may be attached together in a pendent manner or may be fused. The term “aryl” embraces aromatic radicals such as phenyl, naphthyl, tetrahydronapthyl, indane, and biphenyl. The term “heterocyclyl” means a saturated or unsaturated mono- or multi-ring carbocycle wherein one or more carbon atoms is replaced by N, S, P, or O. This includes, for example, structures such as: 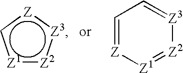
where Z, Z¹, Z², or Z³ is C, S, P, O, or N, with the proviso that one of Z, Z¹, Z², or Z³ is other than carbon, but is not O or S when attached to another Z atom by a double bond or when attached to another O or S atom. Furthermore, the optional substituents are understood to be attached to Z, Z¹, Z², or Z³ only when each is C. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others. The term “heteroaryl” embraces unsaturated heterocyclic radicals. Examples of unsaturated heterocyclic radicals, also termed “heteroaryl” radicals include thienyl, pyrryl, furyl, pyridyl, pyrimidyl, pyrazinyl, pyrazolyl, oxazolyl, isoxazolyl, imidazolyl, thiazolyl, pyranyl, and tetrazolyl. The term also embraces radicals where heterocyclic radicals are fused with aryl radicals. Examples of such fused bicyclic radicals include benzofuran, benzothiophene, and the like. The terms aryl or heteroaryl, as appropriate, include the following structures: 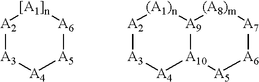
where:
when n=1, m=1 and A₁-A₈ are each CR^{x} or N, A₉ and A₁₀ are carbon;
when n=0, or 1, and m=0, or 1, one of A₂-A₄ and/or A₅-A₇ is optionally S, O, or NR^{x}, and other ring members are CR^{x} or N, with the proviso that oxygen cannot be adjacent to sulfur in a ring. A₉ and A₁₀ are carbon;
when n is greater than or equal to 0, and m is greater than or equal to 0, 1 or more sets of 2 or more adjacent atoms A₁-A₁₀ are sp3 O, S, NR^{x}, CR^{x}R^{y}, or C═(O or S), with the proviso that oxygen and sulfur cannot be adjacent. The remaining A₁-A₈ are CR^{x} or N, and A₉ and A₁₀ are carbon;
when n is greater than or equal to 0, and m is greater than or equal to 0, atoms separated by 2 atoms (i.e., A₁ and A₄) are sp3 O, S, NR^{x}, CR^{x}R^{y}, and remaining A₁-A₈ are independently CR^{x} or N, and A₉ and A₁₀ are carbon.

The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical. The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂—NH₂), which may also be termed an “aminosulfonyl”. The terms “N-alkylsulfamyl” and “N,N-dialkylsulfamyl” denote sulfamyl radicals substituted, respectively, with one alkyl radical, a cycloalkyl ring, or two alkyl radicals. The terms ”N-arylsulfamyl” and “N-alkyl-N-arylsulfamyl” denote sulfamyl radicals substituted, respectively, with one aryl radical, and one alkyl and one aryl radical.

The terms “carboxy” or “carboxyl”, whether used alone or with other terms, such as “carboxyalkyl”, denotes —CO₂—H. The term “carboxyalkyl” embraces radicals having a carboxyradical as defined above, attached to an alkyl radical. The term “carbonyl”, whether used alone or with other terms, such as “alkylcarbonyl”, denotes —(C═O)—. The term “alkylcarbonyl” embraces radicals having a carbonyl radical substituted with an alkyl radical. An example of an “alkylcarbonyl” radical is CH₃—(CO)—. The term “alkylcarbonylalkyl” denotes an alkyl radical substituted with an “alkylcarbonyl” radical. The term “alkoxycarbonyl” means a radical containing an alkoxy radical, as defined above, attached via an oxygen atom to a carbonyl (C═O) radical. Examples of such “alkoxycarbonyl” radicals include (CH₃)₃—C—O—C═O)— and —(O═)C—OCH₃. The term “alkoxycarbonylalkyl” embraces radicals having “alkoxycarbonyl”, as defined above substituted to an alkyl radical. Examples of such “alkoxycarbonylalkyl” radicals include (CH₃)₃C—OC(═O)—(CH₂)₂— and —(CH₂)₂(—O)COCH₃. The terms “amido”, or “carbamyl”, when used alone or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkylradical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl” embraces alkylradicals substituted with an N-alkyl-N-hydroxyamido radical. The term “amidoalkyl” embraces alkyl radicals substituted with amido radicals. The term “aminoalkyl” embraces alkyl radicals substituted with amino radicals. The term “alkylaminoalkyl” embraces aminoalkyl radicals having the nitrogen atom substituted with an alkyl radical. The term “amidino” denotes an —C(—NH)—NH₂ radical. The term “cyanoamidin” denotes an —C(—N—CN)—NH₂ radical. The term “heterocycloalkyl” embraces heterocyclic-substituted alkyl radicals such as pyridylmethyl and thienylmethyl.

The terms “aralkyl”, or “arylalkyl” embrace aryl-substituted alkyl radicals such as benzyl, diphenylmethyl, triphenylmethyl, phenethyl, and diphenethyl. The terms benzyl and phenylmethyl are interchangeable. The term “cycloalkyl” embraces radicals having three to ten carbon atoms, such as cyclopropyl cyclobutyl, cyclopentyl, cyclohexyl, and cycloheptyl. The term “cycloalkenyl” embraces unsaturated radicals having three to ten carbon atoms, such as cylopropenyl, cyclobutenyl, cyclopentenyl, cyclohexenyl, and cycloheptenyl. The term “alkylthio” embraces radicals containing a linear or branched alkyl radical, of one to ten carbon atoms, attached to a divalent sulfur atom. An example of “alkylthio” is methylthio, (CH₃—S—). The term “alkylsulfinyl” embraces radicals containing a linear or branched alkyl radical, of one to ten carbon atoms, attached to a divalent —S(—O)— atom. The terms “N-alkylamino” and “N,N-dialkylamino” denote amino groups which have been substituted with one alkyl radical and with two alkyl radicals, respectively.

The term “acyl”, whether used alone, or within a term such as “acylamino”, denotes a radical provided by the residue after removal of hydroxyl from an organic acid. The term “acylamino” embraces an amino radical substituted with an acyl group. An examples of an “acylamino” radical is acetylamino (CH₃—C(═O)—NH—).

In the naming of substituent groups for general chemical structures, the naming of the chemical components of the group is typically from the terminal group-toward the parent compound unless otherwise noted, as discussed below. In other words, the outermost chemical structure is named first, followed by the next structure in line, followed by the next, etc. until the structure that is connected to the parent structure is named. For example, a substituent group having a structure such as: 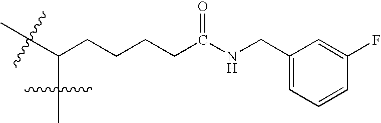
may be referred to generally as a “haloarylalkylaminocarboxylalkyl”. An example of one such group would be fluorophenylmethylcarbamylpentyl. The bonds having wavy lines through them represent the parent structure to which the alkyl is attached.

Substituent groups may also be named by reference to one or more “R” groups. The structure shown above would be included in a description, such as, “—C₁-C₆-alkyl-COR^{u}, where R^{u} is defined to include —NH—C₁-C₄-alkylaryl-R^{y}, and where R^{y} is defined to include halo. In this scheme, atoms having an “R” group are shown with the “R” group being the terminal group (i.e., furthest from the parent). In a term such as “C(R^{x})₂”, it should be understood that the two R^{x} groups can be the same, or they can be different if Rx is defined as having more than one possible identity.

Examples of chromene Cox-2 inhibitors that are suitable for use with the methods and compositions of the present invention include any one or more of:
6-nitro-2-trifluoromethyl-2H-1-benzopyran-3-carboxylic acid; 6-chloro-8-methyl-2-trifluoromethyl-2H-1-benzopyran-3-carboxylic acid; (S)-6-chloro-7-(1,1-dimethylethyl)-2-trifluoromethyl-2H-1-benzopyran-3-carboxylic acid; 2-trifluoromethyl-2H-naphthol[2,3-b]pyran-3-carboxylic acid; 6-chloro-7-(4-nitrophenoxy)-2-(trifluoromethyl)-2H-1-benzopyran-3-carboxylic acid; (S)-6,8-dichloro-2-(trifluoromethyl)-2H-1-benzopyran-3-carboxylic acid; 6-chloro-2-(trifluoromethyl)-4-phenyl-2H-1-benzopyran-3-carboxylic acid; 6-(4-hydroxybenzoyl)-2-(trifluoromethyl)-2H-1-benzopyran-3-carboxylic acid; 2-(trifluoromethyl)-6-[(trifluoromethyl)thiol]-2H-1-benzothiopyran-3-carboxylic acid; 6,8-dichloro-2-trifluoromethyl-2H-1-benzothiopyran-3 carboxylic acid; 6-(1,1-dimethylethyl)-2-(trifluoromethyl)-2H-1-benzothiopyran-3-carboxylic acid; 6,7-difluoro-1,2-dihydro-2-(trifluoromethyl)-3-quinolinecarboxylic acid; 6-chloro-1,2-dihydro-1-methyl-2-(trifluoromethyl)-3-quinolinecarboxylic acid; 6-chloro-2-(trifluoromethyl)-1,2-dihydro[1,8]naphthyridine-3-carboxylic acid; (S)-6-chloro-1,2-dihydro-2-(trifluoromethyl)-3-quinolinecarboxylic acid; (2S)-6,8-dimethyl-2-(trifluoromethyl)-2H-chromene-3-carboxylic acid; (2S)-8-ethyl-6-(trifluoromethoxy)-2-(trifluoromethyl)-2H-chromene-3-carboxylic acid; (2S)-6-chloro-5,7-dimethyl-2-(trifluoromethyl)-2H-chromene-3-carboxylic acid; 6-chloro-2-trifluoromethyl-2 H-1-benzopyran-3-carboxylic acid; 6-chloro-7-methyl-2-trifluoromethyl-2H-1-benzopyran-3-carboxylic acid; 8-(1-methylethyl)-2-trifluoromethyl-2H-1-benzopyran-3-carboxylic acid; 6-chloro-7-(1,1-dimethylethyl)-2-trifluoromethyl-2H-1-benzopyran-3-carboxylic acid; 6-chloro-8-(1-methylethyl)-2-trifluoromethyl-2H-1-benzopyran-3-carboxylic acid; 2-trifluoromethyl-3H-naphthopyran-3-carboxylic acid; 7-(1,1-dimethylethyl)-2-trifluoromethyl-2H-1-benzopyran-3-carboxylic acid; 6-bromo-2-trifluoromethyl-2H-1-benzopyran-3-carboxylic acid; 8-chloro-2-trifluoromethyl-2H-1-benzopyran-3-carboxylic acid; 6-trifluoromethoxy-2-trifluoromethyl-2H-1-benzopyran-3-carboxylic acid; 5,7-dichloro-2-trifluoromethyl-2H-1-benzopyran-3-carboxylic acid; 8-phenyl-2-trifluoromethyl-2H-1-benzopyran-3-carboxylic acid; 7,8-dimethyl-2-trifluoromethyl-2H-1-benzopyran-3-carboxylic acid; 6,8-bis(dimethylethyl)-2-trifluoromethyl-2H-1-benzopyran-3-carboxylic acid; 7-(1-methylethyl)-2-trifluoromethyl-2H-1-benzopyran-3-carboxylic acid; 7-phenyl-2-trifluoromethyl-2H-1-benzopyran-3-carboxylic acid; 6-chloro-7-ethyl-2-trifluoromethyl-2H-1-benzopyran-3-carboxylic acid; 6-chloro-8-ethyl-2-trifluoromethyl-2H-1-benzopyran-3-carboxylic acid; 6-chloro-7-phenyl-2-trifluoromethyl-2H-1-benzopyran-3-carboxylic acid; 6,7-dichloro-2-trifluoromethyl-2H-1-benzopyran-3-carboxylic acid; 6,8-dichloro-2-trifluoromethyl-2H-1-benzopyran-3-carboxylic acid; 2-trifluoromethyl-3H-naptho[2,1-b]pyran-3-carboxylic acid; 6-chloro-8-methyl-2-trifluoromethyl-2H-1-benzopyran-3-carboxylic acid; 8-chloro-6-methyl-2-trifluoromethyl-2H-1-benzopyran-3-carboxylic acid; 8-chloro-6-methoxy-2-trifluoromethyl-2H-1-benzopyran-3-carboxylic acid; 6-bromo-8-chloro-2-trifluoromethyl-2H-1-benzopyran-3-carboxylic acid; 8-bromo-6-fluoro-2-trifluoromethyl-2H-1-benzopyran-3-carboxylic acid; 8-bromo-6-methyl-2-trifluoromethyl-2H-1-benzopyran-3-carboxylic acid; 8-bromo-5-fluoro-2-trifluoromethyl-2H-1-benzopyran-3-carboxylic acid; 6-chloro-8-fluoro-2-trifluoromethyl-2H-1-benzopyran-3-carboxylic acid; 6-bromo-8-methoxy-2-trifluoromethyl-2H-1-benzopyran-3-carboxylic acid; 6-[[(phenylmethyl)amino]sulfonyl]-2-trifluoromethyl-2H-1-benzopyran-3-carboxylic acid; 6-[(dimethylamino)sulfonyl]-2-trifluoromethyl-2H-1-benzopyran-3-carboxylic acid; 6-[(methylamino)sulfonyl]-2-trifluoromethyl-2H-1-benzopyran-3-carboxylic acid; 6-[(4-morpholino)sulfonyl]-2-trifluoromethyl-2H-1-benzopyran-3-carboxylic acid; 6-[(1,1-dimethylethyl)aminosulfonyl]-2-trifluoromethyl-2H-1-benzopyran-3-carboxylic acid; 6-[(2-methylpropyl)aminosulfonyl]-2-trifluoromethyl-2H-1-benzopyran-3-carboxylic acid; 6-methylsulfonyl-2-trifluoromethyl-2H-1-benzopyran-3-carboxylic acid; 8-chloro-6-[[(phenylmethyl)amino]sulfonyl]-2-trifluoromethyl-2H-1-benzopyran-3-carboxylic acid; 6-phenylacetyl-2-trifluoromethyl-2H-1-benzopyran-3-carboxylic acid; 6,8-dibromo-2-trifluoromethyl-2H-1-benzopyran-3-carboxylic acid; 8-chloro-5,6-dimethyl-2-trifluoromethyl-2H-1-benzopyran-3-carboxylic acid; 6,8-dichloro-(S)-2-trifluoromethyl-2H-1-benzopyran-3-carboxylic acid; 6-benzylsulfonyl-2-trifluoromethyl-2H-1-benzopyran-3-carboxylic acid; 6-[[N-(2-furylmethyl)amino]sulfonyl]-2-trifluoromethyl-2H-1-benzopyran-3-carboxylic acid; 6-[[N-(2-phenylethyl)amino]sulfonyl]-2-trifluoromethyl-2H-1-benzopyran-3-carboxylic acid; 6-iodo-2-trifluoromethyl-2H-1-benzopyran-3-carboxylic acid; 7-(1,1-dimethylethyl)-2-pentafluoroethyl-2H-1-benzopyran-3-carboxylic acid; 6-chloro-2-trifluoromethyl-2H-1-benzothiopyran-3-carboxylic acid; 6-chloro-2-trifluoromethyl-2H-1-benzopyran-3-carboxylic acid; (S)-6-chloro-2-trifluoromethyl-2H-1-benzopyran-3-carboxylic acid; 6-chloro-7-(1,1-dimethylethyl)-2-trifluoromethyl-2H-1-benzopyran-3-carboxylic acid; (S)-6-chloro-7-(1,1-dimethylethyl)-2-(trifluoromethyl)-2H-1-benzopyran-3-carboxylic acid; 6-trifluoromethoxy-2-trifluoromethyl-2H-1-benzopyran-3-carboxylic acid; (S)-6-trifluoromethoxy-2-trifluoromethyl-2 H-1-benzopyran-3-carboxylic acid; 6-formyl-2-(trifluoromethyl)-2H-1-benzopyran-3-carboxylic acid; 6-(difluoromethyl)-2-(trifluoromethyl)-2H-1-benzopyran-3-carboxylic acid; 6,8-dichloro-7-methyl-2-(trifluoromethyl)-2H-1-benzopyran-3-carboxylic acid; 6,8-dichloro-2-trifluoromethyl-2H-1-benzopyran-3-carboxylic acid; (S)-6,8-dichloro-2-(trifluoromethyl)-2H-1-benzopyran-3-carboxylic acid; 6-chloro-1,2-dihydro-2-(trifluoromethyl)-3-quinolinecarboxylic acid; (S)-6-chloro-1,2-dihydro-2-(trifluoromethyl)-3-quinolinecarboxylic acid; 6,8-dichloro-1,2-dihydro-2-(trifluoromethyl)-3-quinolinecarboxylic acid; 7-(1,1-dimethylethyl)-2-trifluoromethyl-2H-1-benzopyran-3-carboxylic acid; 6,7-dichloro-2-trifluoromethyl-2H-1-benzopyran-3-carboxylic acid; 5,6-dichloro-2-(trifluoromethyl)-2H-1-benzopyran-3-carboxylic acid; 2,6-bis(trifluoromethyl)-2H-1-benzopyran-3-carboxylic acid; 5,6,7-trichloro-2-(trifluoromethyl)-2H-1-benzopyran-3-carboxylic acid; 6,7,8-trichloro-2-(trifluoromethyl)-2H-1-benzopyran-3-carboxylic acid; 6-iodo-1,2-dihydro-2-(trifluoromethyl)-3-quinolinecarboxylic acid; 6-bromo-1,2-dihydro-2-(trifluoromethyl)-3-quinolinecarboxylic acid; 6-chloro-7-methyl-2-(trifluoromethyl)-2H-1-benzothiopyran-3-carboxylic acid; 6,8-dichloro-2-trifluoromethyl-2H-1-benzothiopyran-3-carboxylic acid; prodrugs thereof, salts thereof; isomers thereof; and/or mixtures thereof.

Further examples of preferred chromene Cox-2 inhibitors include (S)-6-chloro-7-(1,1-dimethylethyl)-2-(trifluoromethyl)-2H-1-benzopyran-3-carboxylic acid, (2S)-6,8-dimethyl-2-(trifluoromethyl)-2H-chromene-3-carboxylic acid, (2S)-6-chloro-8-methyl-2-(trifluoromethyl)-2H-chromene-3-carboxylic acid, (2S)-8-ethyl-6-(trifluoromethoxy)-2-(trifluoromethyl)-2H-chromene-3-carboxylic acid, (S)-6,8-dichloro-2-(trifluoromethyl)-2H-1-benzopyran-3-carboxylic acid, (2S)-6-chloro-5,7-dimethyl-2-(trifluoromethyl)-2H-chromene-3-carboxylic acid, prodrugs thereof, salts thereof, isomers thereof, and/or mixtures thereof.

In another embodiment of the invention, the Cox-2 inhibitor can be selected from the class of tricyclic Cox-2 selective inhibitors represented by the general structure of formula II: 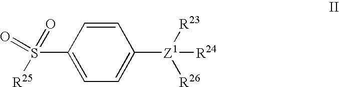
wherein:
Z¹ is selected from the group consisting of partially unsaturated or unsaturated heterocyclyl and partially unsaturated or unsaturated carbocyclic rings;
R²³ is optionally present and if present is selected from an R²⁶ group;
R²⁴ is selected from the group consisting of heterocyclyl, cycloalkyl, cycloalkenyl and aryl, wherein R is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R²⁵ is selected from the group consisting of methyl or amino; and
R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; prodrugs thereof; salts thereof; isomers thereof; and/or mixtures thereof.

In one embodiment of the invention, the tricyclic Cox-2 selective inhibitor comprises at least one compound chosen from celecoxib, parecoxib, deracoxib, valdecoxib, lumiracoxib, etoricoxib, rofecoxib, tilmacoxib, cimicoxib, imrecoxib, prodrugs thereof, salts thereof, isomers thereof, and/or mixtures thereof.

In another embodiment of the invention, the Cox-2 selective inhibitor represented by the above Formula II is chosen from those compounds, illustrated in Table 1, which includes celecoxib (B-1), valdecoxib (B-2), deracoxib (B-3), rofecoxib (B-4), etoricoxib (MK-663; B-5), tilmacoxib (JTE-522) (B-6), cimicoxib (B-7), prodrugs thereof, salts thereof, isomers thereof, and/or mixtures thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-9 (U.S. Pat. No. 5,840,924); compound B-10 (WO 00/25779); imrecoxib (BAP-909) (4-(4-methane-sulfonyl-phenyl-)-1-propyl-3-p-tolyl-1,5-dihydropyrrol-2-one); cimicoxib (4-[4-chloro-5-(3-fluoro-4-methoxyphenyl)-1H-imidzol-1-yl]benzenesulfonamide—CAS RN 265114-23-6); tilmacoxib (4-(4-cyclohexyl-2-methyloxazol-5-yl)-2-fluorobenzenesulfonamide—JTE-522, CAS 180200-68-4); and etoricoxib (CAS RN 202409-33-4, MK-663, SC-86218, and in WO 98/03484).

TABLE 1
Examples of Tricyclic Cox-2 Selective Inhibitors

| Compound | Common | Chemical |
|---|---|---|
| No. | name | name |
| B-1 | celecoxib | 4-[5-(4-methylphenyl)-3- |
| | | (trifluoromethyl)-1H- |
| | | pyrazol-1-yl] benzenesulfonamide |
| B-2 | valdecoxib | 4-(5-methyl-3-phenyl-4-isoxazolyl) |
| | | benzenesulfonamide |
| B-3 | deracoxib | 4- [3-(difluoromethyl)-5-(3- |
| | | fluoro-4-methoxyphenyl)-1H-pyrazol-1- |
| | | yl]benzenesulfonamide |
| B-4 | rofecoxib | 4-[4-(methylsulfonyl)phenyl]- |
| | | 3-phenyl-2(5H)-furanone |
| B-5 | etoricoxib | 2,3′-bipyridine, 5-chloro-6′- |
| | | methyl-3-[4-[methylsulfonyl]phenyl]-; |
| | | or [2] 5-chloro-6′-methyl-3- |
| | | [p-[methylsulfonyl]phenyl]-2,3′- |
| | | bipyridine |
| B-6 | tilmacoxib | 4-(4-cyclohexyl-2-methyloxazol-5-yl)-2- |
| | | fluorobenzenesulfonamide |
| B-7 | cimicoxib | 4-[4-chloro-5-(3-fluoro-4-methoxyphenyl)- |
| | | 1H-imidzol-1-yl]benzenesulfonamide |
| B-8 | parecoxib | N-[[p-(5-methyl-3-phenyl-4-isox- |
| | | azolyl)phenyl]sulfonyl]propionamide |

In yet another embodiment of the invention, the Cox-2 selective inhibitor is selected from the group consisting of celecoxib, valdecoxib, rofecoxib, etoricoxib, tilmacoxib, cimicoxib, etoricoxib, deracoxib, parecoxib, prodrugs thereof, salts thereof, isomers thereof, and/or mixtures thereof. Even more preferred still is that the Cox-2 selective inhibitor is celecoxib.

In another embodiment, the tricyclic Cox-2 selective inhibitor, parecoxib (B-8), N-[[4-(5-methyl-3-phenyl-4-isoxazolyl)phenyl]sulfonyl]-; or (2) N-[[p-(5-methyl-3-phenyl-4-isoxazolyl)phenyl]sulfonyl]propionamide; CAS No.198470-84-7 (See, U.S. Pat. No. 5,932,598), which is a therapeutically effective prodrug of the tricyclic Cox-2 selective inhibitor valdecoxib, compound B-2, (See, U.S. Pat. No. 5,633,272), may be advantageously employed as the Cox-2 inhibitor of the present invention.

A preferred form of parecoxib is sodium parecoxib, which is available as Dynastat®.

Another tricyclic Cox-2 selective inhibitor useful in the present invention is the compound ABT-963, having the structure: 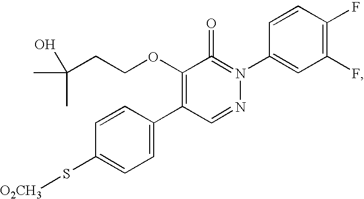
which has been previously described in International Publication Number WO 00/24719.

In a further embodiment of the invention, the Cox-2 inhibitor can be selected from the class of phenylacetic acid derivative Cox-2 selective inhibitors represented by the general structure of formula III: 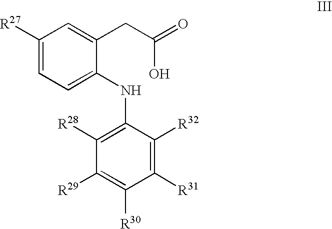
wherein:
R²⁷ is methyl, ethyl, or propyl;
R²⁸ is chloro or fluoro;
R²⁹ is hydrogen, fluoro, or methyl;
R³⁰ is hydrogen, fluoro, chloro, methyl, ethyl, methoxy, ethoxy or hydroxyl;
R³¹ is hydrogen, fluoro, or methyl; and
R³² is chloro, fluoro, trifluoromethyl, methyl, or ethyl, provided that R²⁸, R²⁹, R³⁰ and R³¹ are not all fluoro when R²⁷ is ethyl and R³⁰ is H.

An exemplary phenylacetic acid derivative Cox-2 selective inhibitor that is described in WO 99/11605 is a compound that has the structure shown in formula III,

wherein:

R²⁷ is ethyl;
R²⁸ and R³⁰ are chloro;
R²⁹ and R³¹ are hydrogen; and
R³² is methyl.

Another phenylacetic acid derivative Cox-2 selective inhibitor is a compound that has the structure shown in formula III,

wherein:

R²⁷ is propyl;
R²⁸ and R³⁰ are chloro;
R²⁹ and R³¹ are methyl; and
R³² is ethyl.

Another phenylacetic acid derivative Cox-2 selective inhibitor that is disclosed in WO 02/20090 is a compound that is referred to as COX-189 (also termed lumiracoxib; CAS Reg. No. 220991-20-8), having the structure shown in formula III,

wherein:

R²⁷ is methyl;
R²⁸ is fluoro;
R³² is chloro; and
R²⁹, R³⁰, and R³¹ are hydrogen.

Compounds having a structure similar to that shown in formula VIII, that can serve as the Cox-2 selective inhibitor of the present invention, are described in U.S. Pat. Nos. 6,451,858, 6,310,099, 6,291,523, and 5,958,978.

In certain aspects of the present invention, the Cox-2 selective inhibitor may be a Cox-2 selective inhibitor that is other than any tricyclic Cox-2 selective inhibitor described by formula II. For example, the Cox-2 selective inhibitor may be a chromene Cox-2 inhibitor, which is a class of Cox-2 selective inhibitor that is other than a tricylic Cox-2 selective inhibitor. Likewise, the Cox-2 selective inhibitor may be any compound described by formula III, such as lumiracoxib, which is other than a tricyclic Cox-2 selective inhibitor. Thus, in some embodiments, the present invention encompasses any Cox-2 selective inhibitor that is other than a tricyclic Cox-2 selective inhibitor that is described by formula II, whether such a Cox-2 selective inhibitor is now known or later developed.

In other aspects of the present invention, the Cox-2 selective inhibitor may be at least one compound or class of compounds chosen from Table 2, isomers thereof, salts thereof, and/or mixtures thereof. However, the present invention should not be construed as being limited to any particular one of the Cox-2 selective inhibitors described herein. Indeed, it should be understood that the present invention encompasses any compound that can be shown to act as an inhibitor of the Cox-2 enzyme, whether such a compound is now known, later developed, or even later recognized as having Cox-2 inhibitory activity.

TABLE 2
Additional Cox-2 Selective Inhibitors

| | | Trade | | | | |
|---|---|---|---|---|---|---|
| No. | Generic Name/Compound Name | Name(s) | Drug Class/Mode of Action | Dose | Manufacturer | Reference |
| B11 | Nimesulide | | | | | |
| B12 | Flosulide | | | | | |
| B13 | NS-398 | | | | | CAS RN 123653-11-2 |
| | N-(2-cyclohexyloxynitrophenyl) | | | | | Yoshimi, N. et al., in |
| | methane sulfonamide | | | | | Japanese J. Cancer |
| | | | | | | Res., 90(4): 406-412 |
| | | | | | | (1999) |
| B14 | L-745337 | | | | | |
| B15 | RWJ-63556 | | | | | Kirchner et al., in J |
| | | | | | | Pharmacol Exp Ther |
| | | | | | | 282, 1094-1101 (1997) |
| B16 | L-784512 | | | | | |
| B17 | N-(2- | | diarylmethylidenefuran | | | U.S. Pat. No. |
| | cyclohexyloxynitrophenyl)methane | | derivatives | | | 6,180,651 |
| | sulfonamide, and (E)-4-[(4- | | | | | |
| | methylphenyl)(tetrahydro-2-oxo-3- | | | | | |
| | furanylidene) | | | | | |
| | methyl]benzenesulfonamide | | | | | |
| B18 | Darbufelone | | | | Pfizer | |
| B19 | CS-502 | | | | Sankyo | |
| B20 | LAS 34475 | | | | Almirall | |
| | | | | | Profesfarma | |
| B21 | LAS 34555 | | | | Almirall | |
| | | | | | Profesfarma | |
| B22 | S-33516 | | | | Servier | |
| B23 | SD 8381 | | | | Pharmacia | U.S. Pat. No. |
| | | | | | | 6,034,256 |
| B24 | BMS-347070 | | | | Bristol Myers | U.S. Pat. No. |
| | | | | | Squibb | 6,180,651 |
| B25 | MK-966 | | | | Merck | |
| B26 | L-783003 | | | | Merck | |
| B27 | T-614 | | | | Toyama | |
| B28 | D-1367 | | | | Chiroscience | |
| B29 | L-748731 | | | | Merck | |
| B30 | CT3 | | | | Atlantic | |
| | | | | | Pharmaceuti- | |
| | | | | | cal | |
| B31 | CGP-28238 | | | | Novartis | |
| B32 | BF-389 | | | | Biofor/Scherer | |
| B33 | GR-253035 | | | | Glaxo | |
| | | | | | Wellcome | |
| B34 | 6-dioxo-9H-purin-8-yl-cinnamic | | | | Glaxo | |
| | acid | | | | Wellcome | |
| B35 | S-2474 | | | | Shionogi | |
| B36 | | | Multibinding compounds | | | U.S. Pat. No. |
| | | | containing from 2 to 10 ligands | | | 6,395,724 |
| | | | covanlently attached to one or | | | |
| | | | more linkers | | | |
| B37 | | | Conjugated linoleic acid | | | U.S. Pat. No. |
| | | | derivatives | | | 6,077,868 |
| B38 | | | Heterocyclic aromatic oxazole | | | U.S. patents 5,994,381 |
| | | | compounds | | | and 6,362,209 |
| B39 | | | Miscellaneous compounds | | | U.S. Pat. Nos. |
| | | | | | | 6,596,736, 6,369,275, |
| | | | | | | 6,127,545, 6,130,334, |
| | | | | | | 6,204,387, 6,071,936, |
| | | | | | | 6,001,843 and |
| | | | | | | 6,040,450 |
| B40 | | | Diarylbenzopyran derivatives | | | U.S. Pat. No. |
| | | | | | | 6,340,694 |
| B41 | | | 1-(4-sulfamylaryl)-3-substituted- | | | U.S. Pat. No. |
| | | | 5-aryl-2-pyrazolines | | | 6,376,519 |
| B42 | | | Heterocycle compounds | | | U.S. Pat. No. |
| | | | | | | 6,153,787 |
| B43 | | | 2,3,5-trisubstituted pyridines | | | U.S. Pat. No. |
| | | | | | | 6,046,217 |
| B44 | | | Diaryl bicyclic heterocycles | | | U.S. Pat. No. |
| | | | | | | 6,329,421 |
| B45 | | | Salts of 5-amino or substituted | | | U.S. Pat. No. |
| | | | amino 1,2,3-triazole compounds | | | 6,239,137 |
| B46 | | | Pyrazole derivatives | | | U.S. Pat. 6,136,831 |
| B47 | | | Substituted derivatives of | | | U.S. Pat. 6,297,282 |
| | | | benzosulphonamides | | | |
| B48 | 3-phenyl-4-(4- | | Phenyl heterocycles | | | U.S. Pat. Nos. |
| | (methylsulfonyl)phenyl)-2-(2H)- | | | | | 5,474,995 and |
| | furanone | | | | | 6,239,173 |
| B49 | | | Bicycliccarbonyl indole | | | U.S. Pat. No. |
| | | | compounds | | | 6,303,628 |
| B50 | | | Benzimidazole compounds | | | U.S. Pat. No. |
| | | | | | | 6,310,079 |
| B51 | | | Indole compounds | | | U.S. Pat. No. |
| | | | | | | 6,300,363 |
| B52 | | | Aryl phenylhydrazides | | | U.S. Pat. No. |
| | | | | | | 6,077,869 |
| B53 | | | 2-aryloxy, 4-aryl furan-2-ones | | | U.S. Pat. No. |
| | | | | | | 6,140,515 |
| B54 | | | Bisaryl compounds | | | U.S. Pat. No. |
| | | | | | | 5,994,379 |
| B55 | | | 1,5-diarylpyrazoles | | | U.S. Pat. No. |
| | | | | | | 6,028,202 |
| B56 | | | 2-substituted imidazoles | | | U.S. Pat. No. |
| | | | | | | 6,040,320. |
| B57 | | | 1,3- and 2,3-diarylcycloalkano | | | U.S. Pat. No. |
| | | | and cycloalkeno pyrazoles | | | 6,083,969 |
| B58 | | | Esters derived from | | | U.S. Pat. No. |
| | | | indolealkanols and novel amides | | | 6,306,890 |
| | | | derived from indolealkylamides | | | |
| B59 | | | Pyridazinone compounds | | | U.S. Pat. No. |
| | | | | | | 6,307,047 |
| B60 | | | Benzosulphonamide derivatives | | | U.S. Pat. No. |
| | | | | | | 6,004,948 |
| B61 | | | Methanesulfonyl-biphenyl | | | U.S. Pat. No. |
| | | | derivatives | | | 6,583,321 |
| B62 | | | 1H-indole derivatives | | | U.S. Pat. No. |
| | | | | | | 6,599,929 |
| B63 | N-(2-hydroxyethyl)-4-[5-(4- | | Certain prodrugs of Cox-2 | | | U.S. Pat. Nos. |
| | methylphenyl)-3-(trifluoromethyl)- | | inhibitors | | | 6,436,967 and |
| | 1H-pyrazol-1- | | | | | 6,613,790 |
| | yl]benzenesulfonamide, N,N-bis(2- | | | | | |
| | hydroxyethyl)-4-[5-(4- | | | | | |
| | methylphenyl)-3-(trifluoromethyl)- | | | | | |
| | 1H-pyrazol-1- | | | | | |
| | yl]benzenesulfonamide | | | | | |
| B64 | | | Sulfamoylheteroaryl pyrazole | | | U.S. Pat. No. |
| | | | compounds | | | 6,583,321 |
| B65 | | | Heteroaryl substituted amidinyl | | | U.S. Pat. No. |
| | | | and imidazolyl compounds | | | 6,555,563 |
| B66 | | | Substituted hydroxamic acid | | | U.S. Pat. Nos. |
| | | | derivatives | | | 6,432,999, 6,512,121, |
| | | | | | | and 6,515,014 |
| B67 | | | Pyrazolopyridine compounds | | | U.S. Pat. No. |
| | | | | | | 6,498,166 |
| B68 | | | 4,5-diaryl-3(2H)-furanone | | | U.S. Pat. No. |
| | | | derivatives | | | 6,492,416 |
| B69 | | | 2-phenyl-1,2-benzisoselenazol- | | | U.S. Pat. No. |
| | | | 3(2H)-one derivatives and 2- | | | 6,492,416 |
| | | | phenylcarbomyl-phenylselenyl | | | |
| | | | derivatives | | | |
| B70 | | | Pyrones | | | U.S. Pat. No. |
| | | | | | | 6,465,509 |
| B71 | | | Organically synthesized or | | | U.S. Published |
| | | | purified from plant sources, free- | | | Application No. |
| | | | B-ring flavanoids | | | 2003/0165588 |
| B72 | | | Heterocyclo-alkylsulfonyl | | | European Patent |
| | | | pyrazoles | | | Application No. EP |
| | | | | | | 1312367 |
| B73 | | | 2-phenylpyran-4-one derivatives | | | U.S. Pat. No. |
| | | | | | | 6,518,303 |
| B74 | | | Sulfonylphenylpyrazoles | | | U.S. Pat. No. |
| | | | | | | 6,472,416 |
| B75 | | | 2,3-diaryl-pyrazolo[1,5- | | | U.S. Pat. No. |
| | | | b]pyridazines | | | 6,451,794 |
| B76 | | | (Methylsulfonyl)phenyl | | | U.S. Pat. Nos. |
| | | | furanones | | | 6,169,188, 6,020,343, |
| | | | | | | and 5,981,576 |
| B77 | | | Diaryl-2-(5H)-furanones | | | U.S. Pat. No. |
| | | | | | | 6,222,048 |
| B78 | | | 3,4-diaryl-2-hydroxy-2,5- | | | U.S. Pat. No. |
| | | | dihydrofurans | | | 6,057,319 |
| B79 | | | Carbocyclic sulfonamides | | | U.S. Pat. No. |
| | | | | | | 6,046,236 |
| B80 | | | Oxazole derivatives | | | U.S. Pat. Nos. |
| | | | | | | 6,002,014 and |
| | | | | | | 5,945,539 |
| B81 | | | C-nitroso compounds | | | U.S. Pat. Nos. |
| | | | | | | 6,359,182 and |
| | | | | | | 6,538,116 |
| B82 | | | Substituted pyridines | | | U.S. Published |
| | | | | | | Application No. |
| | | | | | | 2003/0065011 |
| B83 | | | Substituted indole derivatives | | | U.S. Published |
| | | | | | | Application No. |
| | | | | | | 2003/0207897 |
| B84 | meloxicam | | | | | CAS registry number |
| | | | | | | 71125-38-7 |
| B85 | RS 57067 | | | | | CAS registry number |
| | 6-[[5-(4-chlorobenzoyl)-1,4- | | | | | 179382-91-3 |
| | dimethyl-1H-pyrrol-2-yl]methyl]- | | | | | |
| | 3(2H)-pyridazinone | | | | | |

Examples of specific compounds that are useful as Cox-2 selective inhibitors include, without limitation:
8-acetyl-3-(4-fluorophenyl)-2-(4-methylsulfonyl)phenyl-imidazo(1,2-a)pyridine; 5,5-dimethyl-4-(4-methylsulfonyl)phenyl-3-phenyl-2-(5H)-furanone; 5-(4-fluorophenyl)-1-[4-(methylsulfonyl)phenyl]-3-(trifluoromethyl)pyrazole; 4-(4-fluorophenyl)-5-[4-(methylsulfonyl)phenyl]-1-phenyl-3-(trifluoromethyl)pyrazole; 4-(5-(4-chlorophenyl)-3-(4-methoxyphenyl)-1H-pyrazol-1-yl)benzenesulfonamide; 4-(3,5-bis(4-methylphenyl)-1H-pyrazol-1-yl) benzenesulfonamide; 4-(5-(4-chlorophenyl)-3-phenyl-1H-pyrazol-1-yl)benzenesulfonamide; 4-(3,5-bis(4-methoxyphenyl)-1H-pyrazol-1-yl)benzenesulfonamide; 4-(5-(4-chlorophenyl)-3-(4-methylphenyl)-1H-pyrazol-1-yl)benzenesulfonamide; 4-(5-(4-chlorophenyl)-3-(4-nitrophenyl)-1H-pyrazol-1-yl)benzenesulfonamide; 4-(5-(4-chlorophenyl)-3-(5-chloro-2-thienyl)-1H-pyrazol-1-yl)benzenesulfonamide; 4-(4-chloro-3,5-diphenyl-1H-pyrazol-1-yl)benzenesulfonamide; 4-[5-(4-chlorophenyl)-3-(trifluoromethyl)-1H-pyrazol-1-yl]benzenesulfonamide; 4-[5-phenyl-3-(trifluoromethyl)-1H-pyrazol-1-yl]benzenesulfonamide; 4-[5-(4-fluorophenyl)-3-(trifluoromethyl)-1H-pyrazol-1-yl]benzenesulfonamide; 4-[5-(4-methoxyphenyl)-3-(trifluoromethyl)-1H-pyrazol-1-yl]benzenesulfonamide; 4-[5-(4-chlorophenyl)-3-(difluoromethyl)-1H-pyrazol-1-yl]benzenesulfonamide; 4-[5-(4-methylphenyl)-3-(trifluoromethyl)-1H-pyrazol-1-yl]benzenesulfonamide; 4-[4-chloro-5-(4-chlorophenyl)-3-(trifluoromethyl)-1H-pyrazol-1-yl]benzenesulfonamide; 4-[3-(difluoromethyl)-5-(4-methylphenyl)-1H-pyrazol-1-yl]benzenesulfonamide; 4-[3-(difluoromethyl)-5-phenyl-1H-pyrazol-1-yl]benzenesulfonamide; 4-[3-(difluoromethyl)-5-(4-methoxyphenyl)-1H-pyrazol-1-yl]benzenesulfonamide; 4-[3-cyano-5-(4-fluorophenyl)-1H-pyrazol-1-yl]benzenesulfonamide; 4-[3-(difluoromethyl)-5-(3-fluoro-4-methoxyphenyl)-1H-pyrazol-1-yl]benzenesulfonamide; 4-[5-(3-fluoro-4-methoxyphenyl)-3-(trifluoromethyl)-1H-pyrazol-1-yl]benzenesulfonamide; 4-[4-chloro-5-phenyl-1H-pyrazol-1-yl]benzenesulfonamide; 4-[5-(4-chlorophenyl)-3-(hydroxymethyl)-1H-pyrazol-1-yl]benzenesulfonamide; 4-[5-(4-(N,N-dimethylamino)phenyl)-3-(trifluoromethyl)-1H-pyrazol-1-yl]benzenesulfonamide; 5-(4-fluorophenyl)-6-[4-(methylsulfonyl)phenyl]spiro[2.4]hept-5-ene; 4-[6-(4-fluorophenyl)spiro[2.4]hept-5-en-5-yl]benzenesulfonamide; 6-(4-fluorophenyl)-7-[4-(methylsulfonyl)phenyl]spiro[3.4]oct-6-ene; 5-(3-chloro-4-methoxyphenyl)-6-[4-(methylsulfonyl)phenyl]spiro[2.4]hept-5-ene; 4-[6-(3-chloro-4-methoxyphenyl)spiro[2.4]hept-5-en-5-yl]benzenesulfonamide; 5-(3,5-dichloro-4-methoxyphenyl)-6-[4-(methylsulfonyl)phenyl]spiro[2.4]hept-5-ene; 5-(3-chloro-4-fluorophenyl)-6-[4-(methylsulfonyl)phenyl]spiro[2.4]hept-5-ene; 4-[6-(3,4-dichlorophenyl)spiro[2.4]hept-5-en-5-yl]benzenesulfonamide; 2-(3-chloro-4-fluorophenyl)-4-(4-fluorophenyl)-5-(4-methylsulfonylphenyl)thiazole; 2-(2-chlorophenyl)-4-(4-fluorophenyl)-5-(4-methylsulfonylphenyl)thiazole; 5-(4-fluorophenyl)-4-(4-methylsulfonylphenyl)-2-methylthiazole; 4-(4-fluorophenyl)-5-(4-methylsulfonylphenyl)-2-trifluoromethylthiazole; 4-(4-fluorophenyl)-5-(4-methylsulfonylphenyl)-2-(2-thienyl)thiazole; 4-(4-fluorophenyl)-5-(4-methylsulfonylphenyl)-2-benzylaminothiazole; 4-(4-fluorophenyl)-5-(4-methylsulfonylphenyl)-2-(1-propylamino)thiazole; 2-[(3,5-dichlorophenoxy)methyl)-4-(4-fluorophenyl)-5-[4-(methylsulfonyl)phenyl]thiazole; 5-(4-fluorophenyl)-4-(4-methylsulfonylphenyl)-2-trifluoromethylthiazole; 1-methylsulfonyl-4-[1,1-dimethyl-4-(4-fluorophenyl)cyclopenta-2,4-dien-3-yl]benzene; 4-[4-(4-fluorophenyl)-1,1-dimethylcyclopenta-2,4-dien-3-yl]benzenesulfonamide; 5-(4-fluorophenyl)-6-[4-(methylsulfonyl)phenyl]spiro[2.4]hepta-4,6-diene; 4-[6-(4-fluorophenyl)spiro[2.4]hepta-4,6-dien-5-yl]benzenesulfonamide; 6-(4-fluorophenyl)-2-methoxy-5-[4-(methylsulfonyl)phenyl]-pyridine-3-carbonitrile; 2-bromo-6-(4-fluorophenyl)-5-[4-(methylsulfonyl)phenyl]-pyridine-3-carbonitrile; 6-(4-fluorophenyl)-5-[4-(methylsulfonyl)phenyl]-2-phenyl-pyridine-3-carbonitrile; 4-[2-(4-methylpyridin-2-yl)-4-(trifluoromethyl)-1H-imidazol-1-yl]benzenesulfonamide; 4-[2-(5-methylpyridin-3-yl)-4-(trifluoromethyl)-1H-imidazol-1-yl]benzenesulfonamide; 4-[2-(2-methylpyridin-3-yl)-4-(trifluoromethyl)-1H-imidazol-1-yl]benzenesulfonamide; 3-[1-[4-(methylsulfonyl)phenyl]-4-(trifluoromethyl)-1H-imidazol-2-yl]pyridine; 2-[1-[4-(methylsulfonyl)phenyl-4-(trifluoromethyl)-1H-imidazol-2-yl]pyridine; 2-methyl-4-[1-[4-(methylsulfonyl)phenyl-4-(trifluoromethyl)-1H-imidazol-2-yl]pyridine; 2-methyl-6-[1-[4-(methylsulfonyl)phenyl-4-(trifluoromethyl)-1H-imidazol-2-yl]pyridine; 4-[2-(6-methylpyridin-3-yl)-4-(trifluoromethyl)-1H-imidazol-1-yl]benzenesulfonamide; 2-(3,4-difluorophenyl)-1-[4-(methylsulfonyl)phenyl]-4-(trifluoromethyl)-1H-imidazole; 4-[2-(4-methylphenyl)-4-(trifluoromethyl)-1H-imidazol-1-yl]benzenesulfonamide; 2-(4-chlorophenyl)-1-[4-(methylsulfonyl)phenyl]-4-methyl-1H-imidazole; 2-(4-chlorophenyl)-1-[4-(methylsulfonyl)phenyl]-4-phenyl-1H-imidazole; 2-(4-chlorophenyl)-4-(4-fluorophenyl)-1-[4-(methylsulfonyl)phenyl]-1H-imidazole; 2-(3-fluoro-4-methoxyphenyl)-1-[4-(methylsulfonyl)phenyl-4-(trifluoromethyl)-1H-imidazole; 1-[4-(methylsulfonyl)phenyl]-2-phenyl-4-trifluoromethyl-1H-imidazole; 2-(4-methylphenyl)-1-[4-(methylsulfonyl)phenyl]-4-trifluoromethyl-1H-imidazole; 4-[2-(3-chloro-4-methylphenyl)-4-(trifluoromethyl)-1H-imidazol-1-yl]benzenesulfonamide; 2-(3-fluoro-5-methylphenyl)-1-[4-(methylsulfonyl)phenyl]-4-(trifluoromethyl)-1H-imidazole; 4-[2-(3-fluoro-5-methylphenyl)-4-(trifluoromethyl)-1H-imidazol-1-yl]benzenesulfonamide; 2-(3-methylphenyl)-1-[4-(methylsulfonyl)phenyl]-4-trifluoromethyl-1H-imidazole; 4-[2-(3-methylphenyl)-4-trifluoromethyl-1H-imidazol-1-yl]benzenesulfonamide; 1-[4-(methylsulfonyl)phenyl]-2-(3-chlorophenyl)-4-trifluoromethyl-1H-imidazole; 4-[2-(3-chlorophenyl)-4-trifluoromethyl-1H-imidazol-1-yl]benzenesulfonamide; 4-[2-phenyl-4-trifluoromethyl-1H-imidazol-1-yl]benzenesulfonamide; 4-[2-(4-methoxy-3-chlorophenyl)-4-trifluoromethyl-1H-imidazol-1-yl]benzenesulfonamide; 1-allyl-4-(4-fluorophenyl)-3-[4-(methylsulfonyl)phenyl]-5-(trifluoromethyl)-1H-pyrazole; 4-[1-ethyl-4-(4-fluorophenyl)-5-(trifluoromethyl)-1H-pyrazol-3-yl]benzenesulfonamide; N-phenyl-[4-(4-luorophenyl)-3-[4-(methylsulfonyl)phenyl]-5-(trifluoromethyl)-1H-pyrazol-1-yl]acetamide; ethyl [4-(4-fluorophenyl)-3-[4-(methylsulfonyl)phenyl]-5-(trifluoromethyl)-1H-pyrazol-1-yl]acetate; 4-(4-fluorophenyl)-3-[4-(methylsulfonyl)phenyl]-1-(2-phenylethyl)-1H-pyrazole; 4-(4-fluorophenyl)-3-[4-(methylsulfonyl)phenyl]-1-(2-phenylethyl)-5-(trifluoromethyl)pyrazole; 1-ethyl-4-(4-fluorophenyl)-3-[4-(methylsulfonyl)phenyl]-5-(trifluoromethyl)-1H-pyrazole; 5-(4-fluorophenyl)-4-(4-methylsulfonylphenyl)-2-trifluoromethyl-1H-imidazole; 4-[4-(methylsulfonyl)phenyl]-5-(2-thiophenyl)-2-(trifluoromethyl)-1H-imidazole; 5-(4-fluorophenyl)-2-methoxy-4-[4-(methylsulfonyl)phenyl]-6-(trifluoromethyl)pyridine; 2-ethoxy-5-(4-fluorophenyl)-4-[4-(methylsulfonyl)phenyl]-6-(trifluoromethyl)pyridine; 5-(4-fluorophenyl)-4-[4-(methylsulfonyl)phenyl]-2-(2-propynyloxy)-6-(trifluoromethyl)pyridine; 2-bromo-5-(4-fluorophenyl)-4-[4-(methylsulfonyl)phenyl]-6-(trifluoromethyl)pyridine; 4-[2-(3-chloro-4-methoxyphenyl)-4,5-difluorophenyl]benzenesulfonamide; 1-(4-fluorophenyl)-2-[4-(methylsulfonyl)phenyl]benzene; 5-difluoromethyl-4-(4-methylsulfonylphenyl)-3-phenylisoxazole; 4-[3-ethyl-5-phenylisoxazol-4-yl]benzenesulfonamide; 4-[5-difluoromethyl-3-phenylisoxazol-4-yl]benzenesulfonamide; 4-[5-hydroxymethyl-3-phenylisoxazol-4-yl]benzenesulfonamide; 4-[5-methyl-3-phenyl-isoxazol-4-yl]benzenesulfonamide; 1-[2-(4-fluorophenyl)cyclopenten-1-yl]-4-(methylsulfonyl)benzene; 1-[2-(4-fluoro-2-methylphenyl)cyclopenten-1-yl]-4-(methylsulfonyl) benzene; 1-[2-(4-chlorophenyl)cyclopenten-1-yl]-4-(methylsulfonyl)benzene; 1-[2-(2,4-dichlorophenyl)cyclopenten-1-yl]-4-(methylsulfonyl)benzene; 1-[2-(4-trifluoromethylphenyl)cyclopenten-1-yl]-4-(methylsufonyl)benzene; 1-[2-(4-methylthiophenyl)cyclopenten-1-yl]-4-(methylsulfonyl)benzene; 1-[2-(4-fluorophenyl)-4,4-dimethylcyclopenten-1-yl]-4-(methylsulfonyl)benzene; 4-[2-(4-fluorophenyl)-4,4-dimethylcyclopenten-1-yl]benzenesulfonamide; 1-[2-(4-chlorophenyl)-4,4-dimethylcyclopenten-1-yl]-4-(methylsulfonyl)benzene; 4-[2-(4-chlorophenyl)-4,4-dimethylcyclopenten-1-yl]benzenesulfonamide; 4-[2-(4-fluorophenyl)cyclopenten-1-yl]benzenesulfonamide; 4-[2-(4-chlorophenyl)cyclopenten-1-yl]benzenesulfonamide; 1-[2-(4-methoxyphenyl)cyclopenten-1-yl]-4-(methylsulfonyl)benzene; 1-[2-(2,3-difluorophenyl)cyclopenten-1-yl]-4-(methylsulfonyl)benzene; 4-[2-(3-fluoro-4-methoxyphenyl)cyclopenten-1-yl]benzenesulfonamide; 1-[2-(3-chloro-4-methoxyphenyl)cyclopenten-1-yl]-4-(methylsulfonyl)benzene; 4-[2-(3-chloro-4-fluorophenyl)cyclopenten-1-yl]benzenesulfonamide; 4-[2-(2-methylpyridin-5-yl)cyclopenten-1-yl]benzenesulfonamide; ethyl 2-[4-(4-fluorophenyl)-5-[4-(methylsulfonyl) phenyl]oxazol-2-yl]-2-benzyl-acetate; 2-[4-(4-fluorophenyl)-5-[4-(methylsulfonyl)phenyl]oxazol-2-yl]acetic acid; 2-(tert-butyl)-4-(4-fluorophenyl)-5-[4-(methylsulfonyl)phenyl]oxazole; 4-(4-fluorophenyl)-5-[4-(methylsulfonyl)phenyl]-2-phenyloxazole; 4-(4-fluorophenyl)-2-methyl-5-[4-(methylsulfonyl)phenyl]oxazole; 4-[5-(3-fluoro-4-methoxyphenyl)-2-trifluoromethyl-4-oxazolyl]benzenesulfonamide; salts thereof, isomers thereof, and/or mixtures thereof.

Cox-2 inhibitors that are useful in the methods and compositions of present invention can be supplied by any source as long as the Cox-2 inhibitor is pharmaceutically acceptable. Likewise, Cox-2 inhibitors that are useful in the compositions and methods of present invention can be synthesized, for example, according to the description in Example 1. Several Cox-2 inhibitors that are suitable for use with the compositions and methods of the present invention may be synthesized by the methods described in, for example, in U.S. Pat. No. 5,466,823 to Talley, et al.

Various classes of Cox-2 inhibitors useful in the present invention can be prepared as follows. Pyrazoles can be prepared by methods described in WO 95/15316. Pyrazoles can further be prepared by methods described in WO 95/15315. Pyrazoles can also be prepared by methods described in WO 96/03385. Thiophene analogs useful in the present invention can be prepared by methods described in WO 95/00501. Preparation of thiophene analogs is also described in WO 94/15932. Oxazoles useful in the present invention can be prepared by the methods described in WO 95/00501. Preparation of oxazoles is also described in WO 94/27980. Isoxazoles useful in the present invention can be prepared by the methods described in WO 96/25405. Imidazoles useful in the present invention can be prepared by the methods described in WO 96/03388. Preparation of imidazoles is also described in WO 96/03387. Cyclopentene Cox-2 inhibitors useful in the present invention can be prepared by the methods described in U.S. Pat. No. 5,344,991. Preparation of cyclopentene Cox-2 inhibitors is also described in WO 95/00501. Terphenyl compounds useful in the present invention can be prepared by the methods described in WO 96/16934. Thiazole compounds useful in the present invention can be prepared by the methods described in WO 96/03,392. Pyridine compounds useful in the present invention can be prepared by the methods described in WO 96/03392. The preparation of pyridine compounds is also described in WO 96/24,585. Benzopyranopyrazolyl compounds useful in the present invention can be prepared by the methods described in WO 96/09304. Chromene compounds useful in the present invention can be prepared by the methods described in WO 98/47890. Preparation of chromene compounds is also described in WO 00/23433. Chromene compounds can further be prepared by the methods described in U.S. Pat. No. 6,077,850. Preparation of chromene compounds is further described in U.S. Pat. No. 6,034,256. Arylpyridazinones useful in the present invention can be prepared by the methods described in WO 00/24719. Preparation of arylpyridazinones is also described in WO 99/10332. Arylpyridazinones can further be prepared by the methods described in WO 99/10331. 5-Alkyl-2-arylaminophenylacetic acids and derivatives useful in the present invention can be prepared by the methods described in WO 99/11605. Diarylmethylidenefuran derivative Cox-2 selective inhibitors useful in the present invention can be prepared by the methods described in U.S. Pat. No. 6,180,651. The celecoxib used in the compositions and methods of the present invention can be prepared in the manner set forth in U.S. Pat. No. 5,466,823. The valdecoxib used in the compositions and methods of the present invention can be prepared in the manner set forth in U.S. Pat. No. 5,633,272. The parecoxib used in the compositions and methods of the present invention can be prepared in the manner set forth in U.S. Pat. No. 5,932,598. The rofecoxib used in the compositions and methods of the present invention can be prepared in the manner set forth in U.S. Pat. No. 5,474,995. The deracoxib used in the compositions and methods of the present invention can be prepared in the manner set forth in U.S. Pat. No. 5,521,207. The etoricoxib used in the compositions and methods of the present invention can be prepared in the manner set forth in WO 98/03484. The cimicoxib used in the compositions and methods of the present invention can be prepared in the manner set forth in Drugs of the Future, 29(4):325-330 (2004). The imrecoxib used in the compositions and methods of the present invention can be prepared in the manner set forth in Acta Pharmacol Sin, 25(7):927-931 (2004) and Patent Application No. 00105899.1). The meloxicam used in the compositions and methods of the present invention can be prepared in the manner set forth in U.S. Pat. No. 4,233,299. The compound 4-(4-cyclohexyl-2-methyloxazol-5-yl)-2-fluorobenzenesulfonamide used in the compositions and methods of the present invention can be prepared in the manner set forth in U.S. Pat. No. 5,994,381. The compound 2-(3,4-difluorophenyl)-4-(3-hydroxy-3-methylbutoxy)-5-[4-(methylsulfonyl)phenyl]-3(2H)-pyridazinone used in the compositions and methods of the present invention can be prepared in the manner set forth in WO 00/24719. The compound 2-(3,5-difluorophenyl)-3-[4-(methylsulfonyl)phenyl]-2-cyclopenten-1-one used in the compositions and methods of the present invention can be prepared in the manner set forth in EP 863134. The compound 2-[(2-chloro-6-fluorophenyl)amino]-5-methyl-benzeneacetic acid used in the compositions and methods of the present invention can be prepared in the manner set forth in WO 99/11605. The compound N-[2-(cyclohexyloxy)-4-nitrophenyl]methanesulfonamide used in the compositions and methods of the present invention can be prepared in the manner set forth in U.S. Pat. No. 4,885,367. The compound (3Z)-3-[(4-chlorophenyl)[4-(methylsulfonyl)phenyl]methylene]dihydro-2(3H)-furanone used in the compositions and methods of the present invention can be prepared in the manner set forth in U.S. Pat. No. 6,180,651.

Cox-2 inhibitors can also be isolated and purified from natural sources. Cox-2 inhibitors should be of a quality and purity that is conventional in the trade for use in pharmaceutical products.

Another component of the present invention is a cAMP-specific PDE inhibitor. Although any combination of a Cox-2 inhibitor and a cAMP-specific PDE inhibitor is encompassed by the present invention, preferred examples of cAMP-specific PDE inhibitors include those compounds recited in Table 3 and Table 4, or elsewhere herein or isomers thereof, salts thereof, and/or mixtures thereof.

However, the present invention should not be construed as being limited to any particular one of the cAMP-specific PDE inhibitors described herein. It should also be understood that the present invention encompasses any cAMP-specific PDE inhibitor that can be shown to act as a cAMP-specific PDE inhibitor, whether such a compound is now known, later developed, or even later recognized as having in vivo and/or in vitro inhibitory activity against any subtype of a PDE enzyme and that such inhibitory activity leads to an alteration of cAMP levels, as long as the compound is other than a Cox-2 inhibitor.

In one embodiment of the present invention, the cAMP-specific PDE inhibitor comprises at least one compound chosen from a PDE 3 inhibitor, a PDE 4 inhibitor, and mixtures thereof. In another embodiment, the cAMP-specific PDE inhibitor comprises a PDE 3 inhibitor, whether such a compound is now known, later developed, or even later recognized as having PDE 3 inhibitory activity. In a further embodiment, the cAMP-specific PDE inhibitor comprises at least one of the PDE 3 inhibitors listed in Table 3.

TABLE 3
PDE 3 Inhibitors

| | | Trade | Drug | | |
|---|---|---|---|---|---|
| No. | Name | Name(s) | Class | Manufacturer | Reference |
| A1 | Cilostazol | Pletal | PDE 3 inhibitor | Pfizer, Inc. | Liu, Y., et al. Cilostazol (pletal): a dual |
| | 6-[4-(1-Cyclohexyl-1H-tetrazol- | | | | inhibitor of cyclic nucleotide |
| | 5-yl)butoxy]-3,4- | | | | phosphodiesterase type 3 and adenosine |
| | dihydroxycarbostyril | | | | uptake. Cardiovasc Drug Rev, 19: 369-86 |
| | | | | | (2001). |
| A2 | Milrinone | | PDE 3 inhibitor | | Cone, J., et al. Comparison of the effects of |
| | 1,6-dihydro-2-methyl-6-oxo- | | | | cilostazol and milrinone on intracellular |
| | (3,4′-bipyridine)-5-carbonitrile | | | | cAMP levels and cellular function in platelets |
| | | | | | and cardiac cells. J Cardiovasc Pharmacol, |
| | | | | | 34: 497-504 (1999). |
| A3 | Enoximone | | PDE 3 inhibitor | | Abdel-aleem, S., et al. Effects of |
| | 1,3-dihydro-4-methyl-5-[4- | | | | phosphodiesterase inhibitors on glucose |
| | methylthiobenzoyl]-2H- | | | | utilization in isolated cardiac myocytes Mol |
| | imidazol-2-one | | | | Cell Biochem, 180: 129-35 (1998). |
| A4 | Imazodan (Cl-914) | | PDE 3 inhibitor | | Mochizuke, N., et al. Cardiovascular effects |
| | 4,5-dihydro-6-[4-(1H-imidazol- | | | | of NSP-804 and NSP-805, novel cardiotonic |
| | 1-yl)phenyl]-3(2H)-pyridazinone | | | | agents with vasodilator properties J |
| | | | | | Cardiovasc Pharmacol, 21: 983-95 (1993). |
| A5 | Trequinsin | | cAMP-specific | | O'Donnell, et al. Behavioral effects of family- |
| | CAS Number: 78416-81-6 | | PDE inhibitor | | selective inhibitors of cyclic nucleotide |
| | 2-(arylimino)-3-alkyl-9,10- | | | | phosphodiesterases. Pharmacol Biochem |
| | dimethoxy-3,4,6,7-tetrahydro- | | | | Behav, 63: 185-192 (1999). |
| | 2H-pyrimido[6,1- | | | | |
| | a]isoquinolin-4-one | | | | |
| A6 | Olprinone | | PDE 3 inhibitor | | Myou, S. et al. Bronchodilator effect of |
| | 1,2 dihydro-6-methyl-2-oxo-5- | | | | inhaled olprinone, a phosphodiesterase 3 |
| | [imidazo(1,2-a)pyridine | | | | inhibitor, in asthmatic patients. Am J Respir |
| | carbonitrile hydrochloride | | | | Crit Care Med, 160: 817-20 (1999). |
| | monohydrate | | | | |
| A7 | Amrinone | | PDE 3 inhibitor | | Kerttula, T., et al. Amrinone, a |
| | 5-amino-3,4′-bipyridine-6(1H)- | | | | phosphodiesterase III inhibitor, and |
| | one | | | | arachidonic acid metabolism in humans. J |
| | | | | | Cardiovasc Pharmacol. 33: 140-3 (1999). |
| A8 | Indolidan (LY195115) | | cAMP-specific | | Kauffman, R. F., et al. LY195115: a potent, |
| | 3,3-dimethyl-5-(1,4,5,6- | | PDE inhibitor | | selective inhibitor of cyclic nucleotide |
| | tetrahydro-6-oxo-3-pyridazinyl)- | | | | phosphodiesterase located in the |
| | 2-indolinone | | | | sarcoplasmic reticulum. |
| | | | | | Mol Pharmacol, 30: 609-16 (1986). |
| A9 | Siguazodan | | PDE 3 inhibitor | | Christensen and Torphy. Isoenzyme- |
| | N-cyano-N′-methyl-N″-[4- | | | | selective phosphodiesterase inhibitors as |
| | (1,4,5,6-tetrahydro-4-methyl-6- | | | | antiasthmatic agents. Ann Rep Med Chem, |
| | oxo-3-pyridazinyl)phenyl] | | | | 29: 185 (1994). |
| | guanidine | | | | |
| A10 | Zardaverine | | PDE 3 and | | Galvan and Schudt. Actions of the |
| | 6-[4-(Difluoromethoxy)-3- | | PDE 4 inhibitor | | phosphodiesterase inhibitor zardaverine on |
| | methoxyphenyl]-3(2H)- | | | | guinea pig ventricular muscle. Naunyn- |
| | pyridazinone | | | | Schmied Arch Pharmacol, 342: 221 (1990). |
| A11 | Benzafentrine | | PDE 3 and | | Banner, K. H., et al. The effect of selective |
| | | | PDE 4 inhibitor | | phosphodiesterase 3 and 4 isoenzyme |
| | | | | | inhibitors and established anti-asthma drugs |
| | | | | | on inflammatory cell activation. Br J |
| | | | | | Pharmacol. 119: 1255-61 (1996). |
| A12 | 4-methylamino-7-(2,3,4,5- | | PDE 3 and | | Nomoto, Y., et al. Studies on cardiotonic |
| | tetrahydro-5-methyl-3-oxo-6- | | PDE 4 inhibitor | | agents. VI. Synthesis of novel 4,5-dihydro- |
| | pyridazinyl)quinazoline | | | | 3(2H)-pyridazinone derivatives carrying |
| | | | | | some benzoheterocycles at the 6-position. |
| | | | | | Chem Pharm Bull (Tokyo). 39: 352-7 (1991). |
| A13 | Lixazinone (RS-82856) | | PDE 3 and | | Strosberg, A. M., et al. RS-82856, a selective |
| | N-cyclohexyl-N-methyl-4(7-oxy | | PDE 4 inhibitor | | phosphodiesterase inhibitor with inotropic, |
| | 1,2,3,5-tetrahydroimidazol[2,1- | | | | afterload reduction and antithrombotic |
| | b] quinazolin-2-one butyramide | | | | properties. Proc West Pharmacol Soc. 30: 5- |
| | | | | | 10 (1987). |
| A14 | NSP-513 | | PDE 3 inhibitor | | Hirose, H., et al. Antiplatelet and |
| | (R)-4,5-dihydro-5-methyl-6-[4- | | | | antithrombotic effects of a novel selective |
| | (2-propyl-3-oxo-1- | | | | phosphodiesterase 3 inhibitor, NSP-513, in |
| | cyclohexenyl)amino] phenyl- | | | | mice and rats. Jpn J Pharmacol. 82: 188-98 |
| | 3(2H)-pyridazinone | | | | (2000). |
| A15 | Pimobendan | Vetmedin | PDE-3 inhibitor | | Shiga, T., et al. beta-Blocker Therapy |
| | (±)-4,5-dihydro-6-[2-(p- | | and calcium | | Combined with Low-Dose Pimobendan in |
| | methoxyphenyl)-5- | | sensitizer | | Patients with Idiopathic Dilated |
| | benzimidazolyl]-5-methyl- | | | | Cardiomyopathy and Chronic Obstructive |
| | 3(2H)-pyridazinone | | | | Pulmonary Disease: Report on Two Cases. |
| | | | | | Cardiovasc Drugs Ther. 16: 259-63 (2002). |
| A16 | ORG 9935 | | PDE 3 inhibitor | | Santing, R. E., et al. Bronchodilatory and |
| | 4,5-dihydro-6-(5,6- | | | | anti-inflammatory properties of inhaled |
| | dimethoxybenzo[b]thien-2-yl-5- | | | | selective phosphodiesterase inhibitors in a |
| | methyl-3(2H)pyridazinone | | | | guinea pig model of allergic asthma. Eur J |
| | | | | | Pharmacol. 429: 335-44 (2001). |
| A17 | ORG 20241 | | PDE 3 and | | Santing, R. E., et al. Bronchodilatory and |
| | N-hydroxy-4-(3,4- | | PDE 4 inhibitor | | anti-inflammatory properties of inhaled |
| | dimethoxyphenyl)-thiazole-2- | | | | selective phosphodiesterase inhibitors in a |
| | carboximidamide HCl | | | | guinea pig model of allergic asthma. Eur J |
| | | | | | Pharmacol. 429: 335-44 (2001). |
| A18 | Saterinone | | PDE 3 inhibitor | Intravenous | Kieback, A. G., et al. Pharmacokinetics and |
| | | | | infusion over | hemodynamic effects of the |
| | | | | 24 hours at a | phosphodiesterase III inhibitor saterinone in |
| | | | | rate of 1.5 | patients with chronic heart failure. Int J |
| | | | | microgram/kg | Cardiol. 91(2-3): 201-8 (2003). |
| | | | | per min. | |
| A19 | Cilostamide | | PDE 3 inhibitor | | Tarpey, S. B., et al., Phosphodiesterase 3 |
| | | | | | activity is reduced in dog lung following |
| | | | | | pacing-induced heart failure. Am J Physiol |
| | | | | | Lung Cell Mol Physiol. 284(5): L766-73 |
| | | | | | (2003). |

In some embodiments, the cAMP-specific PDE 3 inhibitor comprises at least one compound chosen from cilostazol, milrinone, enoximone, imazodan, trequinsin, olprinone, amrinone, indolidan, siguazodan, zardaverine, benzafentrine, lixazinone, NSP-513, pimobendan, ORG 9935, 4-methylamino-7-(2,3,4,5-tetrahydro-5-methyl-3-oxo-6-pyridazinyl) quinazoline, ORG 20241, saterinone, cilostamide, salts thereof, isomers thereof, prodrugs thereof, and mixtures thereof. In preferred embodiments, the cAMP-specific PDE inhibitor comprises the PDE 3 inhibitor, cilostazol.

In other embodiments of the present invention, the cAMP-specific PDE inhibitor comprises a PDE 4 inhibitor, whether such a compound is now known, later developed, or even later recognized as having PDE 4 inhibitory activity. In a further embodiment, the cAMP-specific PDE inhibitor comprises at least one of the PDE 4 inhibitors listed in Table 4.

TABLE 4
PDE 4 Inhibitors

| | | Trade | Drug | | |
|---|---|---|---|---|---|
| No. | Name | Name(s) | Class | Manufacturer | Reference |
| C1 | Roflumilast | | PDE 4 inhibitor | Altana | Hatzelmann, A. and Schudt, C. Anti- |
| | 3-cyclo-propylmethoxy-4- | | | Pharma | inflammatory and immunomodulatory |
| | difluoromethoxy-N-[3,5-di- | | | | potential of the novel PDE4 inhibitor |
| | chloropyrid-4-yl]-benzamide | | | | roflumilast in vitro. J Pharmacol Exp Ther. |
| | | | | | 297: 267-79 (2001). |
| C2 | Cilomilast | Ariflo ® | PDE 4 inhibitor | GlaxoSmith | Giembycz, M. A. Cilomilast: a second |
| | 4-cyano-4-(3- | | | Kline | generation phosphodiesterase 4 inhibitor for |
| | cyclopentyloxy-4-methoxy- | | | | asthma and chronic obstructive pulmonary |
| | phenyl)cyclohexane | | | | disease. Expert Opin Investig Drugs. |
| | carboxylic acid | | | | 10: 1361-79 (2001). |
| C3 | Etazolate hydrochloride | | selective for | | Chesin, et al. 1-Ethyl-4- |
| | (1-Ethyl-4-[(1- | | PDE4 | | (isopropylidenehydrazinol-H-pyrazolo-(3,4- |
| | methylethylidene) | | | | b)-pyridin-5-carboxylic acid, ethyl ester, |
| | hydrazino]-1H-pyrazolo- | | | | hydrochloride (SQ 20009) - potent new |
| | [3,4-b]-pyridine-5-carboxylic | | | | inhibitor of cyclic 3′5-nucleotide |
| | acid, ethyl ester | | | | phosphodiesterase. Biochem Pharmacol, |
| | | | | | 21: 2443 (1972). |
| C4 | Ro 20-1724 | | selective for | | Nicholson et al. Differential modulation of |
| | 4-(3-Butoxy-4- | | PDE4 | | tissue function and therapeutic potential of |
| | methoxyphenyl) methyl-2- | | | | selective inhibitors of cyclic nucleotide |
| | imidazolidone | | | | phosphodiesterase isoenzymes. TiPS 21: 19 |
| | | | | | (1991). |
| C5 | Rolipram | | Selective | | Kato et al. Rolipram, a cyclic AMP- |
| | 4-(3-(Cyclopentyloxy)-4- | | inhibitor of | | selective phosphodiesterase inhibitor, |
| | methoxyphenyl)pyrrolidin-2- | | PDE4 | | reduces neuronal damage following |
| | one | | | | cerebral ischemia in the gerbil. Eur J |
| | | | | | Pharmacol, 272: 107 (1995). |
| | | | | | Teixeira et al. Phosphodiesterase (PDE)4 |
| | | | | | inhibitors: anti-inflammatory drugs of the |
| | | | | | future? TiPS 18: 164 (1997). |
| C6 | (R)-(−)-Rolipram | | More active | | Schneider et al. Stereospecific binding of |
| | (4R)-4-[3-(Cyclopentyloxy)- | | enantiomer of | | the antidepressant rolipram to brain protein |
| | 4-methoxyphenyl]pyrrolidin- | | the PDE4 | | structures. Eur J Pharmacol, 127: 105 |
| | 2-one | | inhibitor | | (1986). |
| | | | rolipram; 2-10- | | |
| | | | fold more | | |
| | | | potent than the | | |
| | | | S-(+) | | |
| | | | enantiomer. | | |
| C7 | (S)-(+)-Rolipram | | Less active | | Schneider et al. Stereospecific binding of |
| | (4S)-4-[3-(Cyclopentyloxy)- | | enantiomer of | | the antidepressant rolipram to brain protein |
| | 4-methoxyphenyl]pyrrolidin- | | the PDE4 | | structures. Eur J Pharmacol, 127: 105 |
| | 2-one | | inhibitor | | (1986). |
| | | | rolipram. | | |
| C8 | Zardaverine | | Selective for | | Galvan and Schudt. Actions of the |
| | 6-[4-(Difluoromethoxy)-3- | | PDE3 and 4 | | phosphodiesterase inhibitor zardaverine on |
| | methoxyphenyl]-3(2H)- | | (IC₅₀ values are | | guinea pig ventricular muscle. Naunyn- |
| | pyridazinone | | 0.5 and 0.8 μM | | Schmied Arch Pharmacol, 342: 221 (1990). |
| | | | respectively). | | |
| C9 | V11294A | | PDE 4 inhibitor | | Gale, D. D., et al. Pharmacokinetic and |
| | 3-(3-cyclopentyloxy-4- | | | | pharmacodynamic profile following oral |
| | methoxybenzyl)-6- | | | | administration of the phosphodiesterase |
| | ethylamino-8-isopropyl-3H | | | | (PDE)4 inhibitor V11294A in healthy |
| | purine hydrochloride | | | | volunteers. Br J Clin Pharmacol. 54: 478-84 |
| | | | | | (2002). |
| C10 | CDP840 | | PDE 4 inhibitor | Celltech | Perry, M. J., et al. CDP840: a novel inhibitor |
| | R-[+]-4-[2-inverted question | | | | of PDE-4. Cell Biochem Biophys. 29: 113-32 |
| | mark3-cyclopentyloxy-4- | | | | (1998). |
| | methoxyphenyl inverted | | | | |
| | question mark-2- | | | | |
| | phenylethyl]pyridine | | | | |
| C11 | Denbufylline | | PDE 4 inhibitor | | Hadley, A. J., et al. Stimulation of the |
| | 1,3-dibutyl-7-(2-oxopropyl)- | | | | hypothalamo-pituitary-adrenal axis in the rat |
| | 3,7-dihydro-1H-purine-2,6- | | | | by the type 4 phosphodiesterase (PDE-4) |
| | dione | | | | inhibitor, denbufylline. Br J Pharmacol. |
| | | | | | 119: 463-70 (1996). |
| C12 | Mesopram | | PDE 4 inhibitor | | Griffiths, C. E., et al. Randomized |
| | 5-(4-methoxy-3- | | | | comparison of the type 4 |
| | propoxyphenyl)-5-methyl- | | | | phosphodiesterase inhibitor cipamfylline |
| | 1,3-oxazolidin-2-one | | | | cream, cream vehicle and hydrocortisone |
| | | | | | 17-butyrate cream for the treatment of |
| | | | | | atopic dermatitis. Br J Dermatol. 147: 299- |
| | | | | | 307 (2002). |
| C13 | Cipamfylline | | PDE 4 inhibitor | | Loher, F., et al. The specific type 4 |
| | 8-amino-1,3-bis- | | | | phosphodiesterase inhibitor mesopram |
| | cyclopropylmethyl-3,7- | | | | alleviates experimental colitis in mice. J |
| | dihydro-purine-2,6-dione | | | | Pharmacol Exp Ther. 305: 549-56 (2003). |
| C14 | SCH 351591 | | PDE 4 inhibitor | | Billah, M. M., et al. Pharmacology of N-(3,5- |
| | N-(3,5-dichloro-1-oxido-4- | | | | dichloro-1-oxido-4-pyridinyl)-8-methoxy-2- |
| | pyridinyl)-8-methoxy-2- | | | | (trifluoromethyl)-5-quinoline carboxamide |
| | (trifluoromethyl)-5-quinoline | | | | (SCH 351591), a novel, orally active |
| | carboxamide | | | | phosphodiesterase 4 inhibitor. J Pharmacol |
| | | | | | Exp Ther. 302: 127-37 (2002). |
| C15 | SCH 365351 | | PDE 4 inhibitor | | Billah, M. M., et al. Pharmacology of N-(3,5- |
| | N-(3,5-Dichloro-4-pyridinyl)- | | | | dichloro-1-oxido-4-pyridinyl)-8-methoxy-2- |
| | 8-methoxy-2- | | | | (trifluoromethyl)-5-quinoline carboxamide |
| | (trifluoromethyl)-5-quinoline | | | | (SCH 351591), a novel, orally active |
| | carboxamide | | | | phosphodiesterase 4 inhibitor. J Pharmacol |
| | | | | | Exp Ther. 302: 127-37 (2002). |
| C16 | L-791,943 | | PDE 4 inhibitor | | Guay, D., et al. Discovery of L-791,943: a |
| | | | | | potent, selective, non emetic and orally |
| | | | | | active phosphodiesterase-4 inhibitor. Bioorg |
| | | | | | Med Chem Lett. 12: 1457-61 (2002). |
| C17 | 7-Benzylamino-6-chloro-2- | | cAMP-specific | | Wagner, B. et al. 7-Benzylamino-6-chloro- |
| | piperazino-4-pyrrolidino- | | PDE inhibitor | | 2-piperazino-4-pyrrolidino-pteridine, a |
| | pteridine | | | | potent inhibitor of cAMP-specific |
| | | | | | phosphodiesterase, enhancing nuclear |
| | | | | | protein binding to the CRE consensus |
| | | | | | sequence in human tumour cells. Biochem |
| | | | | | Pharmacol. 63: 659-68 (2002). |
| C18 | Piclamilast (RP 73401) | | PDE 4 inhibitor | Rhône- | Corbel, M. et al. The selective |
| | N-(3,5-dichloropyrid-4-yl)-3- | | | Poulenc | phosphodiesterase 4 inhibitor RP 73-401 |
| | cyclopentyloxy-4- | | | Rorer | reduced matrix metalloproteinase 9 activity |
| | methoxybenzamide | | | | and transforming growth factor-beta release |
| | | | | | during acute lung injury in mice: the role of |
| | | | | | the balance between Tumor necrosis factor- |
| | | | | | alpha and interleukin-10. J Pharmacol Exp |
| | | | | | Ther. 301: 258-65 (2002). |
| C19 | NVP-ABE171 | | PDE 4 inhibitor | | Trifilieff, A., et al. Pharmacological profile of |
| | 4-(8-benzo[1,2,5]oxadiazol- | | | | a novel phosphodiesterase 4 inhibitor, 4-(8- |
| | 5-yl-[1,7]naphthyridin-6-yl)- | | | | benzo[1,2,5]oxadiazol-5-yl- |
| | benzoic acid | | | | [1,7]naphthyridin-6-yl)-benzoic acid (NVP- |
| | | | | | ABE171), a 1,7-naphthyridine derivative, |
| | | | | | with anti-inflammatory activities. J |
| | | | | | Pharmacol Exp Ther. 301: 241-8 (2002). |
| C20 | 4-(8-benzo[1,2,5]oxadiazol- | | PDE 4D | | Hersperger, R., et al. Synthesis of 4-(8- |
| | 5-yl-[1,7]naphthyridine-6-yl)- | | inhibitor | | benzo[1,2,5]oxadiazol-5-yl- |
| | benzoic acid | | | | [1,7]naphthyridine-6-yl)-benzoic acid: a |
| | | | | | potent and selective phosphodiesterase |
| | | | | | type 4D inhibitor. Bioorg Med Chem Lett. |
| | | | | | 12: 233-5 (2002). |
| C21 | YM976 | | PDE 4 inhibitor | | Aoki, M, et al. A novel phosphodiesterase |
| | 4-(3-chlorophenyl)-1,7- | | | | type 4 inhibitor, YM976 (4-(3-chlorophenyl)- |
| | diethylpyrido[2,3- | | | | 1,7-diethylpyrido[2,3-d]pyrimidin-2(1H)- |
| | d]pyrimidin-2(1H)-one | | | | one), with little emetogenic activity. J |
| | | | | | Pharmacol Exp Ther. 295: 255-60 (2000). |
| C22 | KF19514 | | PDE 4 inhibitor | | Manabe, H., et al. Anti-inflammatory and |
| | 5-phenyl-3-(3- | | | | bronchodilator properties of KF19514, a |
| | pyridyl)methyl-3H- | | | | phosphodiesterase 4 and 1 inhibitor. Eur J |
| | imidazo[4,5- | | | | Pharmacol. 332: 97-107 (1997). |
| | c][1,8]naphthyridin-4 (5H)- | | | | |
| | one | | | | |
| C23 | Arofylline | | PDE 4 inhibitor | Almirall | Ferrer, L, et al. Clinical anti-inflammatory |
| | 3-(4-Chlorophenyl)-3,7- | | | | efficacy of arofylline, a new selective |
| | dihydro-1-propyl-1H-purine- | | | | phosphodiesterase-4 inhibitor, in dogs with |
| | 2,6-dione; (2) 3-(p- | | | | atopic dermatitis. Vet Pec. 745: 191-4 |
| | Chlorophenyl)-1- | | | | (1999). |
| | propylxanthine | | | | |
| C24 | XT-44 | | PDE 4 inhibitor | | Waki, Y., et al. Effects of XT-44, a |
| | 1-n-butyl-3-n-propylxanthine | | | | phosphodiesterase 4 inhibitor, in |
| | | | | | osteoblastgenesis and osteoclastgenesis in |
| | | | | | culture and its therapeutic effects in rat |
| | | | | | osteopenia models. Jpn J Pharmacol. |
| | | | | | 79: 477-83 (1999). |
| C25 | T-440 | | cAMP-specific | | Sugahara M., et al. An efficient synthesis of |
| | 6,7-Diethoxy-1-[1-(2- | | PDE inhibitor | | the anti-asthmatic agent T-440: a selective |
| | methoxyethyl)-2-oxo-1,2- | | | | N-alkylation of 2-pyridone. Chem Pharm |
| | dihydropyridin-4- | | | | Bull (Tokyo). 48: 589-91 (2000). |
| | yl]naphthalene-2,3- | | | | |
| | dimethanol | | | | |
| C26 | Atizoram (CP-80633) | | PDE 4 inhibitor | Pfizer | Cohan, V. L., et al. In vitro pharmacology of |
| | (2′S)5-[3-(2′- | | | | the novel phosphodiesterase type 4 |
| | exobicyclo[2.2.1]- | | | | inhibitor, CP-80633. J Pharmacol Exp Ther. |
| | heptyloxy)4-methoxyphenyl] | | | | 278: 1356-61 (1996). |
| | tetrahydro-2(1H)-primidone | | | | |
| C27 | Tibenelast (LY 186655) | | PDE 4 inhibitor | Ely Lilly | Ho, P. P., et al. Cardiovascular effect and |
| | 5,6,- | | | | stimulus-dependent inhibition of superoxide |
| | diethoxybenzo(b)thiophene- | | | | generation from human neutrophils by |
| | 2-carboxylic acid | | | | tibenelast, 5,6-diethoxybenzo(b)thiophene- |
| | | | | | 2-carboxylic acid, sodium salt (LY186655). |
| | | | | | Biochem Pharmacol. 40: 2085-92 (1990). |
| C28 | D-4418 | | PDE 4 inhibitor | Chiroscience/ | |
| | | | | Schering- | |
| | | | | Plough | |
| C29 | V-11294A | | PDE 4 inhibitor | Napp/Purdue | |
| | | | | Pharma | |
| C30 | Cl 1018 | | PDE 4 inhibitor | Jouveinal/ | |
| | | | | Park-Davis | |
| C31 | D-22888 | | PDE 4 inhibitor | Asta Medica | |
| C32 | N-(3,5-dichloropyridin-4-yl)- | | PDE 4 inhibitor | Arzneimittel- | U.S. Pat. No. 6,545,158 |
| | 2-[1-(4-fluorobenzyl)-5- | | | werk Dresden | |
| | hydroxyindol-3-yl]-2- | | | GmbH | |
| | oxoacetamide | | | | |
| C33 | 1,2,4-triazolo(4,3- | | PDE 4 | Almirall | U.S. Pat. No. 6,407,108 |
| | b)pyrido(3,2-d)pyridazine | | inhibitors | Prodesfarma, | |
| | derivatives | | | S.A. | |
| C34 | Diazepinoindoles | | PDE 4 | Pfizer, Inc.; | U.S. Pat. No. 6,544,983; 6,239,130; |
| | | | inhibitors | Jouveinal | 5,972,927 |
| | | | | (′927) | |
| C35 | Heterosubstituted pyridine | | PDE 4 | Merck Frosst | U.S. Pat. Nos. 6,316,472; 6,200,993; |
| | derivatives | | inhibitors | Canada & Co. | 6,180,650 |
| C36 | Hydroxyindoles | | PDE 4 | Arzneimittel- | U.S. Pat. No. 6,251,923 |
| | | | inhibitors | werk Dresden | |
| | | | | GmbH | |
| C37 | N-(3,5-Dichloro-1-oxido- | | PDE 4 inhibitor | Merck Frosst | U.S. Pat. No. 6,448,274 |
| | pyridin-4-yl)-4- | | | Canada & Co. | |
| | difluoromethoxy-3- | | | | |
| | cyclopropylmeth- | | | | |
| | oxybenzamide | | | | |
| C38 | Thiazolyl-acid amide | | PDE 4 | Pfizer, Inc. | U.S. Pat. No. 6,559,168 |
| | derivatives | | inhibitors | | |
| C39 | Novel compounds | | PDE 4 | SmithKline | U.S. Pat. No. 5,552,438 |
| | | | inhibitors | Beecham | |
| | | | | Corporation | |
| C40 | Cyclohexene-ylidene | | inhibitors of | SmithKline | U.S. Pat. No. 5,605,923 |
| | derivatives | | TNF production | Beecham | |
| | | | and PDE 4 | Corporation | |
| C41 | 2,3-disubstituted pyridine | | PDE 4 | Dainippon | U.S. Pat. No. 6,555,557 |
| | derivatives | | inhibitors | Pharma- | |
| | | | | ceutical | |
| | | | | Company, | |
| | | | | Limited | |
| C42 | Phenanthridine-N-oxides | | PDE 4 | Altana | U.S. Pat. Nos. 6,538,005; 6,534,519 |
| | | | inhibitors | Pharma AG | |
| C43 | Polysubstituted 6- | | PDE 4 | Altana | U.S. Pat. No. 5,534,518 |
| | phenylphenanthridines | | inhibitors | Pharma AG | |
| C44 | PDE IV inhibiting amides | | PDE 4 | Merck Frosst | U.S. Pat. No. 6,436,965 |
| | | | inhibitors | Canada & Co. | |
| C45 | Aryl thiophene derivatives | | PDE 4 | Merck & Co., | U.S. Pat. No. 6,034,089 |
| | | | inhibitors | Inc. | |
| C46 | Aryl furan derivatives | | PDE 4 | Merck & Co., | U.S. Pat. No. 6,020,339 |
| | | | inhibitors | Inc. | |
| C47 | Amides and Imides | | PDE 3 and | Celgene Corp. | U.S. Pat. Nos. 6,518,281; 6,180,644; |
| | | | PDE 4 | | 5,968,945; 5,728,845; 5,703,098 |
| | | | inhibitors | | |
| C48 | Substituted 1,3,4- | | PDE 3 and | Celgene Corp. | U.S. Pat. No. 6,326,388 |
| | oxadiazoles | | PDE 4 | | |
| | | | inhibitors | | |
| C49 | Cyano and carboxy | | PDE 3 and | Celgene Corp. | U.S. Pat. Nos. 6,479,554; 6,262,101; |
| | derivatives of substituted | | PDE 4 | | 6,130,226; 5,929,117 |
| | styrenes | | inhibitors | | |
| C50 | Substituted | | PDE 4 | Celgene Corp. | U.S. Pat. Nos. 6,020,358; 6,011,050 |
| | phenethylsulfones | | inhibitors | | |
| C51 | Nitriles | | PDE 3 and | Celgene Corp. | U.S. Pat. No. 5,728,844 |
| | | | PDE 4 | | |
| | | | inhibitors | | |
| C52 | Succinimide and maleimide | | PDE 3 and | Celgene Corp. | U.S. Pat. No. 5,658,940 |
| | cytokine inhibitors | | PDE 4 | | |
| | | | inhibitors | | |
| C53 | Nicotinamide benzofused- | | PDE4 inhibitors | Pfizer, Inc. | U.S. Published Application No. |
| | heterocyclyl derivatives | | | | 20030186989 |
| C54 | Ether derivatives | | PDE4 inhibitors | Pfizer, Inc | U.S. Published application No. |
| | | | | | 20030027845 |

In some embodiments, the cAMP-specific PDE 4 inhibitor comprises at least one compound chosen from roflumilast, cilomilast, etazolate hydrochloride, Ro 20-1724, rolipram, (R)-(−)-rolipram, (S)-(+)-rolipram, zardaverine, V11294A, CDP840, denbufylline, mesopram, cipamfylline, SCH 351591, SCH 365351, L-791,943, piclamilast, NVP-ABE171, YM976, KF19514, arofylline, XT-44, T-440, atizoram, tibenelast, D-4418, V-1 1294A, Cl 1018, D-22888, 1,2,4-triazolo(4,3-b)pyrido(3,2-d)pyridazine derivatives, diazepinoindoles, heterosubstituted pyridine derivatives, hydroxyindoles, thiazolyl-acid amide derivatives, novel compound, cyclohexene-ylidene derivatives, 2,3-disubstituted pyridine derivatives, phenanthridine-n-oxides, polysubstituted 6-phenylphenanthridines, PDE 4 inhibiting amides, aryl thiophene derivatives, aryl furan derivatives, amides and imides, substituted 1,3,4-oxadiazoles, cyano and carboxy derivatives of substituted styrenes, substituted phenethylsulfones, nitriles, succinimide and maleimide cytokine inhibitors, nicotinamide benzofused-heterocyclyl derivatives, ether derivatives, salts thereof, isomers thereof, prodrugs thereof, and mixtures thereof. In preferred embodiments, the cAMP-specific PDE 4 inhibitor comprises the PDE 4 inhibitor, roflumilast.

In accordance with the present invention, any composition comprising a Cox-2 inhibitor and a cAMP-specific PDE inhibitor may be administered to a subject according to standard routes of drug delivery that are well known to one of ordinary skill in the art.

The Cox-2 inhibitors and cAMP-specific PDE inhibitors can be supplied in the form of a salt, a prodrug, an isomer, a tautomer, a racemic mixture, or in any other chemical form or combination that, under physiological conditions, still provides for inhibition of the Cox-2 enzyme and any physiological function that the cAMP-specific PDE inhibitor may perform. The present invention includes all possible diastereomers as well as their racemic and resolved, enantiomerically pure forms.

The compounds useful in the present invention can have no asymmetric carbon atoms, or, alternatively, the useful compounds can have one or more asymmetric carbon atoms. When the useful compounds have one or more asymmetric carbon atoms, they, therefore, include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention. Also included in the methods, combinations and compositions of the present invention are the tautomeric forms of the described compounds.

Also included in the methods and compositions of the present invention are the prodrugs of the described compounds and the pharmaceutically-acceptable salts thereof.

As used herein, the term “prodrug” refers to drug precursor compounds which, following administration to a subject and subsequent absorption, are converted to an active species in vivo via some process, such as a metabolic process. A nonlimiting example of a “prodrug” that will be useful in the methods, combinations and compositions of the present invention is the Cox-2 inhibitor, parecoxib (N-[[4-(5-methyl-3-phenyl-4-isoxazolyl)phenyl]sulfonyl]propanamide).

The Cox-2 inhibitors and cAMP-specific PDE inhibitors that are useful in the present invention can be of any purity or grade, as long as the preparation is of a quality suitable for pharmaceutical use. The Cox-2 inhibitors and cAMP-specific PDE inhibitors can be provided in pure form, or they can be accompanied with impurities or commonly associated compounds that do not affect their physiological activity or safety.

The term “pharmaceutically acceptable” is used adjectivally herein to mean that the modified noun is appropriate for use in a pharmaceutical product.

The compounds of the present invention can also be supplied in the form of a pharmaceutically acceptable salt. The terms “pharmaceutically acceptable salt” refer to salts prepared from pharmaceutically acceptable inorganic and organic acids and bases.

Pharmaceutically acceptable inorganic bases include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary salts include aluminum, calcium, lithium, magnesium, potassium, sodium and zinc. Particularly preferred are the ammonium, calcium, magnesium, potassium, and sodium salts.

Salts derived from pharmaceutically acceptable organic non-toxic bases include salts of primary, secondary, and tertiary amines, including in part, trimethylamine, diethylamine, N,N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

Illustrative pharmaceutically acceptable acid addition salts of the compounds of the present invention can be prepared from the following acids, including, without limitation formic, acetic, propionic, benzoic,succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, β-hydroxybutyric, galactaric and galacturonic acids. Exemplary pharmaceutically acceptable salts include the salts of hydrochloric acid and trifluoroacetic acid.

All of the above salts can be prepared by those skilled in the art by conventional means from the corresponding compound of the present invention. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p.1418, the disclosure of which is hereby incorporated by reference only with regards to the disclosures of pharmaceutically acceptable salts.

In another embodiment of the present invention, the combination of a Cox-2 inhibitor and a cAMP-specific PDE inhibitor can be provided in a “pharmaceutically acceptable carrier” or “pharmaceutically acceptable excipient”, both of which are used interchangeably herein, to form a pharmaceutical composition.

Pharmaceutically acceptable carriers and excipients include, but are not limited to, physiological saline, Ringer's solution, phosphate solution or buffer, buffered saline and other carriers known in the art. Pharmaceutical compositions may also include stabilizers, anti-oxidants, colorants, and diluents. Pharmaceutically acceptable carriers and excipients are chosen such that side effects from the pharmaceutical compound are minimized and the performance of the compound is not canceled or inhibited to such an extent that treatment is ineffective.

The pharmaceutically acceptable carrier can also be selected on the basis of the desired route of administration of the compound. For example, in a preferred embodiment the carrier is suitable for oral administration. In some embodiments, the composition includes a carrier or additional agent that is suitable for promoting delivery of the compound to the brain. Examples of such carriers include those disclosed in U.S. Pat. Nos. 5,604,198; 5,827,819; 5,919,815; 5,955,459; and 5,977,174.

The carrier should be acceptable in the sense of being compatible with the other ingredients of the composition and not be deleterious to the recipient. The carrier can be a solid or a liquid, or both, and is preferably formulated with the compound as a unit-dose composition, for example, a tablet, which can contain from 0.05% to 95% by weight of the active compound.

The pharmaceutical compositions of the invention can be prepared by any of the well-known techniques of pharmacy, for example, by admixing the components.

The Cox-2 inhibitors or the cAMP-specific PDE inhibitors can be administered to a subject by any conventional means and route available for use in conjunction with pharmaceuticals, either as individual therapeutic compounds or as a combination of therapeutic compounds or as independent multiple pharmaceutical compositions.

In the combination therapies, administration of two or more of the therapeutic agents useful in the methods and compositions of the present invention may take place sequentially in separate formulations, or may be accomplished by simultaneous administration in a single formulation or in a separate formulation. The formulation may be in the form of a bolus, or in the form of aqueous or non-aqueous isotonic sterile injection solutions or suspensions. For example, the therapeutic compounds which make up the combination therapy may be a combined dosage form or in separate dosage forms intended for substantially simultaneous oral administration. The therapeutic compounds, which make up the combination therapy, may also be administered sequentially, with either therapeutic compound being administered by a regimen calling for two-step ingestion. Thus, a regimen may call for sequential administration of the therapeutic compounds with spaced-apart ingestion of the separate, active agents. The time period between the multiple ingestion steps may range from, for example, a few minutes to several hours to days depending upon the properties of each therapeutic compound such as potency, solubility, bioavailability, plasma half-life and kinetic profile of the therapeutic compound, as well as depending upon the effect of food ingestion and the age and condition of the patient. Circadian variation of the target molecule concentration may also determine the optimal dose interval.

The therapeutic compounds of the combined therapy whether administered simultaneously, substantially simultaneously, or sequentially, may involve a regimen calling for administration of one therapeutic compound by oral route and another therapeutic compound by intravenous route. Whether the therapeutic compounds of the combined therapy are administered enterally or parenterally, separately or together, each therapeutic compound may be contained in a suitable pharmaceutical formulation of any of the pharmaceutically-acceptable excipients, diluents or other formulations components described herein. Thus, the combination of therapeutic compounds may be administered by any combination of, for example, oral/oral, oral/parenteral, or parenteral/parenteral route.

Pharmaceutical compositions according to the present invention include those suitable for enteral (e.g., oral and buccal), inhalation spray, rectal, topical, or parenteral (e.g., subcutaneous, intramuscular, intravenous, intrathecal, intramammary, intramedullary and intradermal injections, or infusion techniques) administration, although the most suitable route in any given case will depend on the nature and severity of the condition being treated and on the nature of the particular compound which is being used.

In most cases, the preferred route of administration is enteral (e.g., orally). Oral administration includes solution, tablets, sustained release capsules, enteric-coated capsules, and syrups. The pharmaceutical composition may be adminsitered in dosage unit formulations containing conventional nontoxic pharmaceutically acceptable carriers, adjuvants, and vehicles as desired. When administered, the pharmaceutical composition may be at or near body temperature.

The compounds of the present invention can be delivered orally either in a solid, in a semi-solid, or in a liquid form. Pharmaceutically acceptable carriers can be in solid dosage forms for the methods of the present invention, which include tablets, capsules, gelcaps, pills, and granules, which can be prepared with coatings and shells, such as enteric coatings and others well known in the art. Liquid dosage forms for oral administration include pharmaceutically acceptable emulsions, solutions, suspensions, syrups, and elixirs.

Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Pharmaceutical compositions suitable for oral administration can be presented in discrete units each containing a predetermined amount of at least one therapeutic compound useful in the present invention; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Syrups and elixirs containing the Cox-2 inhibitor and cAMP-specific PDE inhibitor may be formulated with sweetening agents, for example glycerol, sorbitol, or sucrose. Such formulations may also contain a demulcent, a preservative and flavoring and coloring agents. Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Aqueous suspensions can be produced that contain the active materials in a mixture with excipients suitable for the manufacture of aqueous suspensions. Such excipients include suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents, which may be naturally-occurring phosphatides, for example lecithin, or condensation products of an alkylene oxide with fatty acids, for example, polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, or antioxidants such as ascorbic acid; one or more coloring agents; one or more flavoring agents; and/or one or more sweetening agents, such as sucrose or saccharin. Solutions and suspensions may be prepared from powders or granules having one or more pharmaceutically acceptable carriers or diluents, or a binder such as gelatin or hydroxypropylmethyl cellulose, together with one or more of a lubricant, preservative, surface active or dispersing agent.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

Oral delivery of the combinations of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. For some of the therapeutic compounds useful in the methods, combinations and compositions of the present invention the intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Also encompassed by the present invention is buccal or “sub-lingual” administration, which includes lozenges or a chewable gum comprising the compounds, set forth herein. The compounds can be deposited in a flavored base, usually sucrose, and acacia or tragacanth, and pastilles comprising the compounds in an inert base such as gelatin and glycerin or sucrose and acacia.

The subject method of prescribing a Cox-2 inhibitor and/or cAMP-specific PDE inhibitor and compositions comprising the same can also be administered parenterally, for example, by either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents, which have been mentioned above, or other acceptable agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

Pharmaceutical compositions suitable for parenteral administration can conveniently comprise sterile aqueous preparations of a compound of the present invention. These preparations are preferably administered intravenously, although administration can also be effected by means of subcutaneous, intramuscular, or intradermal injection or by infusion. Such preparations can conveniently be prepared by admixing the compound with water and rendering the resulting solution sterile and isotonic with the blood. Injectable compositions according to the invention will generally contain from 0.1 to 10% w/w of a compound disclosed herein.

Injectable preparations, for example, sterile injectable aqueous or oleaginous suspensions may be formulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The active ingredients may also be administered by injection as a composition wherein, for example, saline, dextrose, or water may be used as a suitable carrier. A suitable daily dose of each active therapeutic compound is one that achieves the same blood serum level as produced by oral administration as described above.

The dose of any of these parenteral compounds can be conveniently administered as an infusion of from about 10 ng/kg body weight to about 10,000 ng/kg body weight per minute. Infusion fluids suitable for this purpose can contain, for example, from about 0.1 ng to about 10 mg, preferably from about 1 ng to about 10 mg per milliliter. Unit doses can contain, for example, from about 1 mg to about 10 g of the compound of the present invention. Thus, ampoules for injection can contain, for example, from about 1 mg to about 100 mg.

Administration of either one or both of the Cox-2 inhibitor and cAMP-specific PDE inhibitor can also be by inhalation, in the form of aerosols or solutions for nebulizers. Therefore, in one embodiment, the Cox-2 inhibitor and cAMP-specific PDE inhibitor are administered by direct inhalation into the respiratory system of a subject for delivery as a mist or other aerosol or dry powder. Delivery of drugs or other active ingredients directly to the subject's lungs provides numerous advantages including, providing an extensive surface area for drug absorption, direct delivery of therapeutic agents to the disease site in the case of regional drug therapy, eliminating the possibility of drug degradation in the subject's intestinal tract (a risk associated with oral administration), and eliminating the need for repeated subcutaneous injections.

Administration of the compositions of the present invention can also be rectally. Pharmaceutical compositions suitable for rectal administration are preferably presented as unit-dose suppositories. These can be prepared by admixing a compound or compounds of the present invention with one or more suitable non-irritating excipients, for example, cocoa butter, synthetic mono- di- or triglycerides, fatty acids and polyethylene glycols that are solid at ordinary temperatures, but liquid at the rectal temperature and will therefore melt in the rectum and release the drug; and then shaping the resulting mixture.

Administration may also be by transvaginal delivery through the use of an intravaginal device. Transvaginal delivery may be desirable for many certain subjects because 10 to 30 times more treatment agent can be delivered transvaginally as can be delivered orally due to the absorption from the vagina, which far exceeds the absorption of drugs from the gastrointestinal tract. Further, vaginal administration generally avoids major problems connected with oral administration, such as gastric and esophageal reflux and ulceration.

Pharmaceutical compositions suitable for topical application to the skin preferably take the form of an ointments, creams, lotions, pastes, gels, sprays, powders, jellies, collyriums, solutions or suspensions, aerosols, or oils. Carriers, which can be used, include petroleum jelly (e.g., Vaseline®), lanolin, polyethylene glycols, alcohols, and combinations of two or more thereof. The active compound or compounds are generally present at a concentration of from 0.1 to 50% w/w of the composition, for example, from 0.5 to 2%.

Other methods for administration of the compounds described herein include dermal patches that relase the medications directly into a subject's skin. Such patches can contain a compound or compounds of the present invention in an optionally buffered, aqueous solution, dissolved and/or dispersed in an adhesive, or dispersed in a polymer. A suitable concentration of the active compound or compounds is about 1% to 35%, preferably about 3% to 15%. As one particular possibility, the compound or compounds can be delivered from the patch by electrotransport or iontophoresis, for example, as described in Pharmaceutical Research 3(6):318 (1986).

A penetration enhancer is an agent used to increase the permeability of the skin to an active agent to increase the rate at which the drug diffuses through the skin and enters the tissues and bloodstream. Thus, in one embodiment of the present invention, a penetration enhancer may be added to a Cox-2 inhibitor and cAMP-specific PDE inhibitor topical composition.

Examples of penetration enhancers suitable for use with the compositions of the present invention include: alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and mixtures thereof.

The compositions of the present invention can optionally be supplemented with additional agents such as, for example, viscosity enhancers, preservatives, surfactants and penetration enhancers.

Viscosity-building agents include, for example, polyvinyl alcohol, polyvinyl pyrrolidone, methylcellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

Preservatives can be optionally employed to prevent microbial contamination of the compositions described herein. Suitable preservatives include, but are not limited to, polyquaternium-1, benzalkonium chloride, thimerosal, chlorobutanol, methyl paraben, propyl paraben, phenylethyl alcohol, edetate disodium, sorbic acid, or other agents known to those skilled in the art. The use of polyquaternium-1 as the antimicrobial preservative is preferred. Typically, such preservatives are employed at a level of from 0.001% to 1.0% by weight.

The solubility of the components of the present compositions may be enhanced by a surfactant or other appropriate co-solvent in the composition. Such co-solvents include polysorbate 20, 60, and 80, polyoxyethylene/polyoxypropylene surfactants (e.g. Pluronic F-68, F-84 and P-103), cyclodextrin, or other agents known to those skilled in the art. Typically, such co-solvents are employed at a level of from 0.01% to 2% by weight.

Pharmaceutically acceptable excipients and carriers encompass all the foregoing and the like. The above considerations concerning effective formulations and administration procedures are well known in the art and are described in standard textbooks. See e.g. Gennaro, A. R., Remington: The Science and Practice of Pharmacy, 20^{th} Edition, (Lippincott, Williams and Wilkins), 2000; Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

The therapeutic compounds which make up the combination therapy may be a combined dosage form or in separate dosage forms intended for substantially simultaneous oral administration. The therapeutic compounds, which make up the combination therapy, may also be administered sequentially, with either therapeutic compound being administered by a regimen calling for two-step ingestion. Thus, a regimen may call for sequential administration of the therapeutic compounds with spaced-apart ingestion of the separate, active agents. The time period between the multiple ingestion steps may range from, for example, a few minutes to several hours to days depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by intravenous route. Whether the therapeutic compounds of the combined therapy are administered enterally or parenterally, separately or together, each such therapeutic compound may be contained in a suitable pharmaceutical formulation of any of the aforementioned pharmaceutically-acceptable excipients, diluents or other formulations components.

The amount of compound in combination that is required to achieve the desired biological effect will, of course, depend on a number of factors such as the specific compound chosen, the use for which it is intended, the mode of administration, and the host to be treated and the clinical condition of the recipient.

For purposes of the present invention, it is preferred that the amount of a Cox-2 inhibitor and the amount of a cAMP-specific PDE inhibitor together comprise an effective amount of the combination of the two treatment agents. Still further preferred is that the amount of the co-therapy with the Cox-2 inhibitor and cAMP-specific PDE inhibitor comprises a therapeutically effective amount of the co-therapy.

Thus, in a preferred embodiment, the present invention provides a method of preventing or treating vascular disorders in a subject comprising administering an amount of a Cox-2 inhibitor and an amount of a cAMP-specific PDE inhibitor wherein the amount of the Cox-2 inhibitor and the amount of the cAMP-specific PDE inhibitor together comprises a therapeutically effective amount.

As used herein, an “effective amount” means the dose or amount to be administered to a subject and the frequency of administration to the subject, which is readily determined by one having ordinary skill in the art, by the use of known techniques and by observing results obtained under analogous circumstances.

In determining the effective amount or dose, a number of factors are considered by the attending diagnostician, including, but not limited to, the potency and duration of action of the compounds used, the nature and severity of the illness to be treated, as well as the sex, age, weight, general health and individual responsiveness of the patient to be treated, and other relevant circumstances.

An vascular disorder symptom is considered ameliorated or improved if any benefit is achieved, no matter how slight. Likewise, an vascular disorder symptom is considered ameliorated or improved if any benefit is achieved, no matter how slight.

A “therapeutically effective amount” is intended to qualify the amount of a Cox-2 inhibitor and a cAMP-specific PDE inhibitor required to treat, prevent or inhibit a vascular disorder.

As used herein, the terms “prophylactically effective” refer to an amount of a Cox-2 inhibitor in combination with a cAMP-specific PDE inhibitor that causes a decrease in the frequency of incidence of a vascular disorder. The term “prophylactic” refers to the prevention of of vascular disorders, whereas the term “therapeutic” refers to the effective treatment of an existing vascular disorder.

It will be appreciated that the amount of the Cox-2 inhibitor and the cAMP-specific PDE inhibitor required for use in the treatment or prevention of a vascular disorder will vary within wide limits and will be adjusted to the individual requirements in each particular case. In general, for administration to adults, an appropriate daily dosage is described herein, although the limits that are identified as being preferred may be exceeded if expedient. The daily dosage can be administered as a single dosage or in divided dosages. For purposes of calculation of dosage amounts, the weight of a normal adult human will be assumed to be 70 kg.

When the term “about” is used herein in relation to a dosage amount of the Cox-2 inhibitor or cAMP-specific PDE inhibitor, it is to be understood to mean an amount that is within ±0.05 mg. By way of example, “about 0.1-10 mg/day” includes all dosages within 0.05 to 10.05 mg/day.

For the purposes of calculating and expressing a dosage rate, all dosages that are expressed herein are calculated on an average amount-per-day basis irrespective of the dosage rate. For example, one 100 mg dosage of a Cox-2 inhibitor taken once every two days would be expressed as a dosage rate of 50 mg/day. Similarly, the dosage rate of an ingredient where 50 mg is taken twice per day would be expressed as a dosage rate of 100 mg/day.

The appropriate dosage level of a Cox-2 inhibitor will generally be from about 0.01 mg per kg to about 140 mg per kg subject body weight per day, which may be administered in single or multiple doses. Preferably, the dosage level will be about 0.1 mg/kg to about 25 mg/kg per day and more preferably about 0.5 mg/kg to about 10 mg/kg per day.

In larger mammals, for example humans, a typical indicated dose of the Cox-2 inhibitor is about 0.5 mg to 7 grams orally per day. A Cox-2 inhibiting compound may be administered on a regimen of several times per day, for example 1 to 4 times per day, preferably once or twice per day.

The amount of the Cox-2 inhibitor that may be combined with the carrier materials to produce a single dosage form will vary depending upon the host treated and the particular mode of administration. For example, a formulation intended for the oral administration of humans may contain from 0.5 mg to 7 g of active agent compounded optionally with an appropriate and convenient amount of carrier material, which may vary from about 5 to about 95 percent of the total composition. Dosage unit forms for the Cox-2 inhibitor will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

In the subject invention, the cAMP-specific PDE inhibitor can be used in any amount that is an effective amount. It is preferred, however, that the amount of the cAMP-specific PDE inhibitor that is administered is within a range of about 0.0001 mg/day per kilogram of the subject to about 100 mg/day/kg. It is more preferred that the amount of the cAMP-specific PDE inhibitor be within a range of about 0.01 mg/day/kg to about 10 mg/day/kg. An amount that is within a range of about 0.01 mg/day/kg to about 5 mg/day/kg, is even more preferred. A total daily dose of a cAMP-specific PDE inhibitor can generally be in the range of from about 0.001 to about 10,000 mg/day in single or divided doses, with preferred levels of between about 0.1 mg to about 1000 mg. By way of example, the cAMP-specific PDE 4 inhibitor, roflumilast is generally dosed at about 0.007 mg/day/kg and the cAMP-specific PDE 3 inhibitor, cilostazol is generally dosed at about 1.4 mg/day/kg. The frequency of dose will depend upon the half-life of the cAMP-specific PDE inhibitor molecule. If the cAMP-specific PDE inhibitor has a short half-life (e.g., from about 2 to 10 hours) it may be necessary to give one or more doses per day. Alternatively, if the half-life is longer (e.g., from about 2 to about 15 days) it may only be necessary to give a dosage once per day, per week, or even once every 1 or 2 months.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages. It is understood, however, that specific dose levels of the therapeutic agents or therapeutic approaches of the present invention for any particular subject depends upon a variety of factors including the activity of the specific compound employed, the age, body weight, general health, sex, and diet of the subject, the time of administration, the rate of excretion, the drug combination, and the severity of the particular disease being treated and form of administration.

Treatment dosages generally may be titrated to optimize safety and efficacy. Typically, dosage-effect relationships from in vitro initially can provide useful guidance on the proper doses for patient administration. Studies in animal models also generally may be used for guidance regarding effective dosages for treatment of vascular disorders in accordance with the present invention. In terms of treatment protocols, it should be appreciated that the dosage to be administered will depend on several factors, including the particular agent that is administered, the route administered, the condition of the particular patient, etc. Generally speaking, one will desire to administer an amount of the compound that is effective to achieve a serum level commensurate with the concentrations found to be effective in vitro. Thus, where a compound is found to demonstrate in vitro activity at, e.g., 10 μM, one will desire to administer an amount of the drug that is effective to provide about a 10 μM concentration in vivo. Determination of these parameters is well within the skill of the art.

Dosages for the combination therapy provided herein may be determined and adjusted based on the efficacy demonstrated in reducing or preventing the symptoms of a vascular disorder. In addition, one of ordinary skill in the art will know how to measure and quantify the presence or absence of vascular disorder symptoms.

Those skilled in the art will also appreciate that dosages may also be determined with guidance from Goodman & Gilman's The Pharmacological Basis of Therapeutics, Ninth Edition (1996), Appendix II, pp. 1707-1711.

It is preferred that the methods and compositions of the present invention are used in the treatment and/or prevention of a vascular disorder in a subject, and in preferred embodiments, the subject is in need of the treatment or prevention of a vascular disorder.

As used herein, the term “subject” includes any subject, and preferably the subject is in need of the prevention or treatment of a vascular disorder. For purposes of prevention, the term “subject” refers to any subject, and preferably is a subject that is at risk for, or is predisposed to, developing a vascular disorder. For purposes of treatment, the term “subject” refers to any subject, and preferably is a subject that is suffering from any symptom of a vascular disorder. As used herein, the terms “subject is in need of the treatment or prevention of a vascular disorder” refer to any subject who is suffering from or is predisposed to any vascular disorder described herein.

The method of the present invention is useful for, but not limited to, the prevention and/or treatment of any vascular disorder and/or vascular disorder-related complication now known or later discovered. As used herein, the terms “vascular disorder” refer to any disorder or complication associated with with the vasculature of a subject including, but are not limited to, diseases of the arteries, including atherosclerosis, peripheral arterial disease, aortic aneurysm, and deep vein thrombosis, bacterial endocarditis, cardiomyopathy, congenital cardiovascular defects, congestive heart failure, rheumatic heart disease, and valvular heart disease, coronary heart disease, heart attack, stroke, transient ischemic attack, peripheral arterial disease, aortic aneurysm, deep vein thrombosi, myocardial ischemia, hypertension, hypotension, heart arrhythmias, including atrial fibrillation and flutter, tachycardia, and ventricular fibrillation, pulmonary hypertension, hypokalemia, vascular rejection, atherosclerosis including cardiac transplant atherosclerosis, myocardial infarction, embolism, thrombosis, including venous thrombosis, angina including unstable angina and angine pectoris, coronary plaque inflammation, ischemia including cardiac and cerebral ischemia, myocardial infarction, cardiac remodeling, cardiac fibrosis, myocardial necrosis, aneurysm, arterial fibrosis, embolism, vascular plaque inflammation, vascular plaque rupture, edema, swelling, fluid accumulation, Bartter's syndrome, myocarditis, arteriosclerosis, calcification (such as vascular calcification and valvar calcification), coronary artery disease, heart failure, congestive heart failure, shock, arrhythmia, left ventricular hypertrophy, angina, diabetic nephropathy, kidney failure, eye damage, migraine headaches, aplastic anemia, cardiac damage, diabetic cardiac myopathy, renal insufficiency, renal injury, renal arteriopathy, peripheral vascular disease, cognitive dysfunction, headache, and inflammation associated with surgical procedures such as vascular grafting including coronary artery bypass surgery, revascularization procedures including angioplasty, stent placement, endarterectomy, or other invasive procedures involving arteries, veins and capillaries.

The methods and compositions of the present invention not only encompass the prevention or treatment of vascular disorders in humans, but also in several animals. For example, many animals also suffer adverse consequences related to vascular disorders. Moreover, many vascular disorders in dogs respond to the same treatment used in humans. Accordingly, besides being useful for humans, the methods and compositions of the present invention also encompass the treatment and prevention of vascular disorders in several nonhumans such as other mammals, including horses, dogs, cats, rats, mice, sheep, pigs, cattle, hamsters, gerbils, and the like. Thus, it is preferred that the subject is an animal, and yet more preferred, the subject is a mammal. Preferably, the mammal is a human.

Finally, in preferred embodiments, the present invention also encompasses a kit comprising one dosage form comprising a Cox-2 inhibitor and a second dosage form comprising a cAMP-specific PDE inhibitor.

In further preferred embodiments, the present invention provides a kit comprising a first dosage form comprising a Cox-2 inhibitor and a second dosage form comprising a cAMP-specific PDE inhibitor, in quantities which comprise a therapeutically effective amount of the compounds for the treatment, prevention or inhibition of a vascular disorder.

The following examples describe embodiments of the invention. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples. In the examples, all percentages are given on a weight basis unless otherwise indicated.

### EXAMPLE 1

This example demonstrates the preparation of celecoxib.

Step 1: Preparation of 1-(4-methylphenyl)-4,4,4-trifluorobutane-1,3-dione.

Following the disclosure provided in U.S. Pat. No. 5,760,068, 4′-Methylacetophenone (5.26 g, 39.2 mmol) was dissolved in 25 mL of methanol under argon and 12 mL (52.5 mmol) sodium methoxide in methanol (25%) was added. The mixture was stirred for 5 minutes and 5.5 mL (46.2 mmol) ethyl trifluoroacetate was added. After refluxing for 24 hours, the mixture was cooled to room temperature and concentrated. 100 mL 10% HCI was added and the mixture extracted with 4×75 mL ethyl acetate. The extracts were dried over MgSO₄, filtered and concentrated to afford 8.47 g (94%) of a brown oil which was carried on without further purification.

Step 2: Preparation of 4-[5-(4-methylphenyl)-3-(trifluoromethyl)-1H-pyrazol-1-yl]benzenesulfonamide.

To the dione from Step 1 (4.14 g, 18.0 mmol) in 75 mL absolute ethanol, 4.26 g (19.0 mmol) 4-sulphonamidophenylhydrazine hydrochloride was added. The reaction was refluxed under argon for 24 hours. After cooling to room temperature and filtering, the reaction mixture was concentrated to afford 6.13 g of an orange solid. The solid was recrystallized from methylene chloride/hexane to give 3.11 g (8.2 mmol, 46%) of the product as a pale yellow solid, having a melting point (mp) of 157°-159°C.; and a calculated composition of C₁₇H₁₄N₃O₂SF₃; C, 53.54; H, 3.70; N, 11.02. The composition that was found by analysis was: C, 53.17; H, 3.81; N, 10.90.

### EXAMPLE 2

This example illustrates the production of a composition containing celecoxib and cilostazol, and of a pharmaceutical composition containing the combination.

Cilostazol can be supplied by any commercial preparation, such as Pletal®. Pletal® is supplied in the form of tablets by Pfizer, Inc, New York, N.Y.

Celecoxib can be prepared as described in Example 1 or, alternatively, can be obtained under the trade name Celebrex®) from Pharmacia Corporation, Peapack, N.J.

A therapeutic composition of the present invention can be produced by intermixing solid or powdered cilostazol (100 g, available as Pletal® from Pfizer, Inc.); and 4-[5-(4-methylphenyl)-3-(trifluoromethyl)-1H-pyrazol-1-yl]benzenesulfonamide (200 g, as produced in Example 1, or as available from Pharmacia Corporation, Peapack, N.J., under the tradename Celebrex®), in a laboratory mill or mixing device suitable for mixing of powders without generating shear force or temperature sufficient to degrade either of the two compounds.

After mixing, the combination of cilostazol and celecoxib forms a therapeutic composition that is sufficient for the production of 1000 human single dose unit, each dose containing 100 mg of cilostazol and 200 mg of celecoxib.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense. In addition, it should be understood that aspects of the various embodiments may be interchanged both in whole or in part.

1. A method for preventing or treating vascular disorders and vascular disorder-related complications in a subject comprising administering to the subject a Cox-2 inhibitor and a cAMP-specific PDE inhibitor.

2. The method according to claim 1, wherein the subject is in need of the prevention or treatment of a vascular disorder and/or a vascular disorder-related complication.

3. The method according to claim 1, wherein the Cox-2 inhibitor comprises a non-steroidal anti-inflammatory drug.

4. The method according to claim 1, wherein the Cox-2 inhibitor comprises a Cox-2 selective inhibitor.

5. The method according to claim 4, wherein the Cox-2 selective inhibitor comprises at least one compound that is chosen from celecoxib, parecoxib, deracoxib, valdecoxib, rofecoxib, etoricoxib, meloxicam, rofecoxib, lumiracoxib, tilmacoxib, cimicoxib, nimesulide, flosulide, darbufelone, RS 57067, T-614, BMS-347070, S-2474, SVT-2016, CT-3, ABT-963, SC-58125, NS-398, L-745337, RWJ-63556, L-784512, CS-502, LAS-34475, LAS-34555, S-33516, SD-8381, PMI-001, 644784, CS-706, PAC-10549, PAC-10649, salts thereof, isomers thereof, prodrugs thereof, and mixtures thereof, and mixtures thereof.

6. The method according to claim 4, wherein the Cox-2 selective inhibitor comprises a tricyclic Cox-2 selective inhibitor.

7. The method according to claim 6, wherein the tricyclic Cox-2 selective inhibitor comprises at least one compound that is chosen from celecoxib, parecoxib, deracoxib, valdecoxib, etoricoxib, tilmacoxib, cimicoxib, prodrugs of any of them, and mixtures thereof.

8. The method according to claim 4, wherein the Cox-2 selective inhibitor comprises at least one compound that is other than a tricyclic Cox-2 selective inhibitor.

9. The method according to claim 8, wherein the Cox-2 selective inhibitor comprises at least one compound chosen from a chromene Cox-2 selective inhibitor, lumiracoxib, RS 57067, NS-398, BMS 347070, ABT-963, SD-8381, PAC-10549, PAC-10649, salts thereof, isomers thereof, prodrugs thereof, and mixtures thereof.

10. The method according to claim 9, wherein the chromene Cox-2 selective inhibitor comprises at least one compound that is chosen from:
(S)-6-chloro-7-(1,1-dimethylethyl)-2-(trifluoromethyl)-2H-1-benzopyran-3-carboxylic acid,
(2S)-6,8-dimethyl-2-(trifluoromethyl)-2H-chromene-3-carboxylic acid,
(2S)-6-chloro-8-methyl-2-(trifluoromethyl)-2H-chromene-3-carboxylic acid,
(2S)-8-ethyl-6-(trifluoromethoxy)-2-(trifluoromethyl)-2H-chromene-3-carboxylic acid,
(S)-6,8-dichloro-2-(trifluoromethyl)-2H-1-benzopyran-3-carboxylic acid,
(2S)-6-chloro-5,7-dimethyl-2-(trifluoromethyl)-2H-chromene-3-carboxylic acid, and mixtures thereof.

11. The method according to claim 1, wherein the cAMP-specific PDE inhibitor comprises at least compound that is chosen from a PDE 3 inhibitor, a PDE 4 inhibitor and mixtures thereof.

12. The method according to claim 1, wherein the cAMP-specific PDE inhibitor comprises a PDE 3 inhibitor.

13. The method according to claim 1, wherein the cAMP-specific PDE inhibitor comprises a PDE 4 inhibitor.

14. The method according to claim 12, wherein the PDE 3 inhibitor comprises at least compound that is chosen from cilostazol, milrinone, enoximone, imazodan, trequinsin, olprinone, amrinone, indolidan, siguazodan, zardaverine, benzafentrine, lixazinone, NSP-513, pimobendan, ORG 9935, 4-methylamino-7-(2,3,4,5-tetrahydro-5-methyl-3-oxo-6-pyridazinyl) quinazoline, ORG 20241, saterinone, cilostamide, salts thereof, isomers thereof, prodrugs thereof, and mixtures thereof.

15. The method according to claim 12, wherein the PDE 3 inhibitor comprises cilostazol.

16. The method according to claim 13, wherein the PDE 4 inhibitor comprises at least compound that is chosen from roflumilast, cilomilast, etazolate hydrochloride, Ro 20-1724, rolipram, (R)-(−)-rolipram, (S)-(+)-rolipram, zardaverine, V1 294A, CDP840, denbufylline, mesopram, cipamfylline, SCH 351591, SCH 365351, L-791,943, piclamilast, NVP-ABE171, YM976, KF19514, arofylline, XT-44, T-440, atizoram, tibenelast, D-4418, V-11294A, Cl 1018, D-22888, 1,2,4-triazolo(4,3-b)pyrido(3,2-d)pyridazine derivatives, diazepinoindoles, heterosubstituted pyridine derivatives, hydroxyindoles, thiazolyl-acid amide derivatives, novel compound, cyclohexene-ylidene derivatives, 2,3-disubstituted pyridine derivatives, phenanthridine-n-oxides, polysubstituted 6-phenylphenanthridines, PDE 4 inhibiting amides, aryl thiophene derivatives, aryl furan derivatives, amides and imides, substituted 1,3,4-oxadiazoles, cyano and carboxy derivatives of substituted styrenes, substituted phenethylsulfones, nitriles, succinimide and maleimide cytokine inhibitors, nicotinamide benzofused-heterocyclyl derivatives, ether derivatives, salts thereof, isomers thereof, prodrugs thereof, and mixtures thereof.

17. The method according to claim 13, wherein the PDE 4 inhibitor comprises roflumilast.

18. The method according to claim 1, wherein the vascular disorder and vascular disorder-related complication is chosen from diseases of the arteries, including atherosclerosis, peripheral arterial disease, aortic aneurysm, and deep vein thrombosis, bacterial endocarditis, cardiomyopathy, congenital cardiovascular defects, congestive heart failure, rheumatic heart disease, and valvular heart disease, coronary heart disease, heart attack, stroke, transient ischemic attack, peripheral arterial disease, aortic aneurysm, deep vein thrombosi, myocardial ischemia, hypertension, hypotension, heart arrhythmias, including atrial fibrillation and flutter, tachycardia, and ventricular fibrillation, pulmonary hypertension, hypokalemia, vascular rejection, atherosclerosis including cardiac transplant atherosclerosis, myocardial infarction, embolism, thrombosis, including venous thrombosis, angina including unstable angina and angine pectoris, coronary plaque inflammation, ischemia including cardiac and cerebral ischemia, myocardial infarction, cardiac remodeling, cardiac fibrosis, myocardial necrosis, aneurysm, arterial fibrosis, embolism, vascular plaque inflammation, vascular plaque rupture, edema, swelling, fluid accumulation, Bartter's syndrome, myocarditis, arteriosclerosis, calcification (such as vascular calcification and valvar calcification), coronary artery disease, heart failure, congestive heart failure, shock, arrhythmia, left ventricular hypertrophy, angina, diabetic nephropathy, kidney failure, eye damage, migraine headaches, aplastic anemia, cardiac damage, diabetic cardiac myopathy, renal insufficiency, renal injury, renal arteriopathy, peripheral vascular disease, cognitive dysfunction, headache, and inflammation associated with surgical procedures such as vascular grafting including coronary artery bypass surgery, revascularization procedures including angioplasty, stent placement, endarterectomy, or other invasive procedures involving arteries, veins and capillaries.

19. A composition comprising at least one Cox-2 inhibitor and at least one cAMP-specific PDE inhibitor.

20. The composition according to claim 19, wherein the Cox-2 inhibitor comprises a non-steroidal anti-inflammatory drug.

21. The composition according to claim 19, wherein the Cox-2 inhibitor comprises a Cox-2 selective inhibitor.

22. The composition according to claim 21, wherein the Cox-2 selective inhibitor comprises at least one compound that is chosen from celecoxib, parecoxib, deracoxib, valdecoxib, rofecoxib, etoricoxib, meloxicam, rofecoxib, lumiracoxib, tilmacoxib, cimicoxib, nimesulide, flosulide, darbufelone, RS 57067, T-614, BMS-347070, S-2474, SVT-2016, CT-3, ABT-963, SC-58125, NS-398, L-745337, RWJ-63556, L-784512, CS-502, LAS-34475, LAS-34555, S-33516, SD-8381, PMI-001, 644784, CS-706, PAC-10549, PAC-10649, salts thereof, isomers thereof, prodrugs thereof, and mixtures thereof, and mixtures thereof.

23. The composition according to claim 21, wherein the Cox-2 selective inhibitor comprises a tricyclic Cox-2 selective inhibitor.

24. The composition according to claim 23, wherein the tricyclic Cox-2 selective inhibitor comprises at least one compound that is chosen from celecoxib, parecoxib, deracoxib, valdecoxib, etoricoxib, tilmacoxib, cimicoxib, prodrugs of any of them, and mixtures thereof.

25. The composition according to claim 23, wherein the Cox-2 selective inhibitor comprises at least one compound that is other than a tricyclic Cox-2 selective inhibitor.

26. The composition according to claim 25, wherein the Cox-2 selective inhibitor comprises at least one compound chosen from a chromene Cox-2 selective inhibitor, lumiracoxib, RS 57067, NS-398, BMS 347070, ABT-963, SD-8381, PAC-10549, PAC-10649, salts thereof, isomers thereof, prodrugs thereof, and mixtures thereof.

27. The composition according to claim 26, wherein the chromene Cox-2 selective inhibitor comprises at least one compound that is chosen from:
(S)-6-chloro-7-(1,1-dimethylethyl)-2-(trifluoromethyl)-2H-1-benzopyran-3-carboxylic acid,
(2S)-6,8-dimethyl-2-(trifluoromethyl)-2H-chromene-3-carboxylic acid,
(2S)-6-chloro-8-methyl-2-(trifluoromethyl)-2H-chromene-3-carboxylic acid,
(2S)-8-ethyl-6-(trifluoromethoxy)-2-(trifluoromethyl)-2H-chromene-3-carboxylic acid,
(S)-6,8-dichloro-2-(trifluoromethyl)-2H-1-benzopyran-3-carboxylic acid,
(2S)-6-chloro-5,7-dimethyl-2-(trifluoromethyl)-2H-chromene-3-carboxylic acid, and mixtures thereof.

28. The composition according to claim 19, wherein the cAMP-specific PDE inhibitor comprises at least compound that is chosen from a PDE 3 inhibitor, a PDE 4 inhibitor and mixtures thereof.

29. The composition according to claim 19, wherein the cAMP-specific PDE inhibitor comprises a PDE 3 inhibitor.

30. The composition according to claim 19, wherein the cAMP-specific PDE inhibitor comprises a PDE 4 inhibitor.

31. The composition according to claim 29, wherein the PDE 3 inhibitor comprises at least compound that is chosen from cilostazol, milrinone, enoximone, imazodan, trequinsin, olprinone, amrinone, indolidan, siguazodan, zardaverine, benzafentrine, lixazinone, NSP-513, pimobendan, ORG 9935, 4-methylamino-7-(2,3,4,5-tetrahydro-5-methyl-3-oxo-6-pyridazinyl) quinazoline, ORG 20241, saterinone, cilostamide, salts thereof, isomers thereof, prodrugs thereof, and mixtures thereof.

32. The composition according to claim 30, wherein the PDE 4 inhibitor comprises at least compound that is chosen from roflumilast, cilomilast, etazolate hydrochloride, Ro 20-1724, rolipram, (R)-(−)-rolipram, (S)-(+)-rolipram, zardaverine, V11294A, CDP840, denbufylline, mesopram, cipamfylline, SCH 351591, SCH 365351, L-791,943, piclamilast, NVP-ABE171, YM976, KF19514, arofylline, XT-44, T-440, atizoram, tibenelast, D-4418, V-11294A, Cl 1018, D-22888, 1,2,4-triazolo(4,3-b)pyrido(3,2-d)pyridazine derivatives, diazepinoindoles, heterosubstituted pyridine derivatives, hydroxyindoles, thiazolyl-acid amide derivatives, novel compound, cyclohexene-ylidene derivatives, 2,3-disubstituted pyridine derivatives, phenanthridine-n-oxides, polysubstituted 6-phenylphenanthridines, PDE 4 inhibiting amides, aryl thiophene derivatives, aryl furan derivatives, amides and imides, substituted 1,3,4-oxadiazoles, cyano and carboxy derivatives of substituted styrenes, substituted phenethylsulfones, nitriles, succinimide and maleimide cytokine inhibitors, nicotinamide benzofused-heterocyclyl derivatives, ether derivatives, salts thereof, isomers thereof, prodrugs thereof, and mixtures thereof.

33. A pharmaceutical composition comprising a Cox-2 inhibitor, a cAMP-specific PDE inhibitor, and a pharmaceutically-acceptable excipient.

34. A kit comprising a first dosage form comprising a Cox-2 inhibitor and a second dosage form comprising a cAMP-specific PDE inhibitor.
